(19) United States
(12) Patent Application Publication
(10) Pub. No.: US 2024/0425470 A1
(43) Pub. Date: Dec. 26, 2024

(54) SUBSTITUTED BENZOFURAN PROPYL AMINE MODULATORS OF MONOAMINERGIC TRANSPORTERS

(71) Applicant: ATAI Life Sciences AG, Berlin (DE)

(72) Inventors: Tanweer A. KHAN, Bridgewater, NJ (US); Glenn SHORT, Scituate, MA (US); Robert B. PERNI, Marlborough, MA (US)

(21) Appl. No.: 18/737,447

(22) Filed: Jun. 7, 2024

Related U.S. Application Data
(60) Provisional application No. 63/472,054, filed on Jun. 9, 2023.

(51) Int. Cl. C07D 305/00; A61K 31/343; C07D 405/12

(52) U.S. Cl. CPC C07D 305/00; A61K 31/343; C07D 405/12

(57) ABSTRACT

Provided herein are compounds of Formula (I) and Formula (II), or a pharmaceutically acceptable salt thereof, wherein R₁, R₂, R₃, R₄, R₅, R₆, R₇, and X are defined herein. Also provided are pharmaceutical compositions comprising a compound of Formula (I) and (II) or a pharmaceutically acceptable salt thereof, and methods of using a compound of Formula (I) and (II) or a pharmaceutically acceptable salt thereof, e.g., in the treatment of a mental disease or disorder.

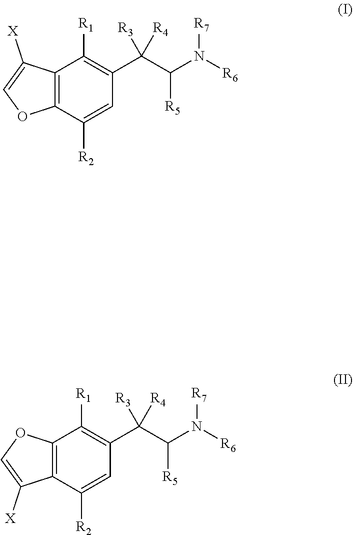

### CROSS-REFERENCE TO RELATED APPLICATIONS

The present application claims priority to U.S. Provisional Application No. 63/472,054, filed Jun. 9, 2023, which is hereby incorporated by reference in its entirety for all purposes.

### BACKGROUND

3,4-Methylenedioxymethamphetamine (MDMA), also known as Ecstasy, is considered the prototype of a class of compounds called entactogens. Structurally, MDMA is a ring-substituted phenethylamine with a chiral center that gives rise to two enantiomeric stereoisomers: S(+)-MDMA and R(−)-MDMA. Typically, effects associated with the S-enantiomer resemble those of psychostimulants and are primarily mediated by dopaminergic and noradrenergic pathways, including increases in motor activity and euphoria, whereas effects associated with the R-enantiomer induces qualitative effects similar to classical psychedelics, such as ego-dissolution and perceptive alterations, mediated by serotonergic pathways, including direct 5-HT2A receptor agonism (Murnane et al., 2009).

Harnessing the biological activity of MDMA in an effective therapeutic treatment has been somewhat limited. This is due at least in part to a problematic pharmacokinetic profile related to non-proportional dose-dependency upon administration in humans. Studies have demonstrated that MDMA is metabolized to three main metabolites identified as 3,4-methylenedioxyamphetamine (MDA), 4-hydroxy-3-methoxymethamphetamine (HMMA), and 4-hydroxymethamphetamine (HMA), which are found in the plasma in different proportions, depending on the concentration of the drug administered. At doses around 50-100 mg, HMMA predominates, while at doses between 125-150 mg MDMA predominates, indicating a possible saturation of its own metabolic pathways. The main hepatic enzymes involved in MDMA metabolism, as identified from in vitro experiments in human liver microsomes, are CYP1A2, CYP2D6, and CYP3A4. Furthermore, stereochemistry appears to play a role in the non-renal pharmacokinetics of MDMA, with the S-enantiomer having a shorter half-life, lower peak plasma concentrations, and increased clearance. Green, A. R., Mechan, A. O., Elliott, J. M., O'Shea, E. & Colado, M. I. The Pharmacology and Clinical Pharmacology of 3,4-Methylenedioxymethamphetamine (MDMA, “Ecstasy”). Pharmacol Rev 55, 463-508 (2003).

Accordingly, novel approaches that improve the pharmacokinetics of MDMA and MDA, and derivatives thereof, are needed in order to take advantage of the wide ranging pharmacologic properties of these compounds in treating various diseases and conditions.

### SUMMARY

In embodiments, the disclosure provides a compound of Formula (I):

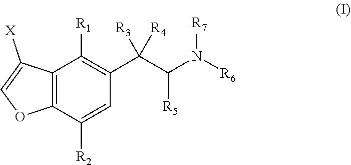

or a pharmaceutically acceptable salt thereof, wherein
X is halogen, deuterium, OH, OR₉, or CF₃;
   wherein R₉ is hydrogen, deuterium, halogen, alkyl, heteroalkyl, haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
   R₃ and R₄ together are carbonyl; or
   R₃ and R₄ together with the carbon atom to which they are attached form a cycloalkyl;
R₅ is hydrogen, deuterium, halogen, alkyl, deuterated alkyl, heteroalkyl, haloalkyl, O-alkyl, O-heteroalkyl, or O-haloalkyl; and
R₆ and R₇ are independently hydrogen, deuterium, alkyl, allyl, haloalkyl, cycloalkyl, or heterocyclyl, wherein the alkyl, deuterated alkyl, haloalkyl, cycloalkyl, or heterocyclyl are independently optionally substituted; or
   one of R₆ or R₇ is

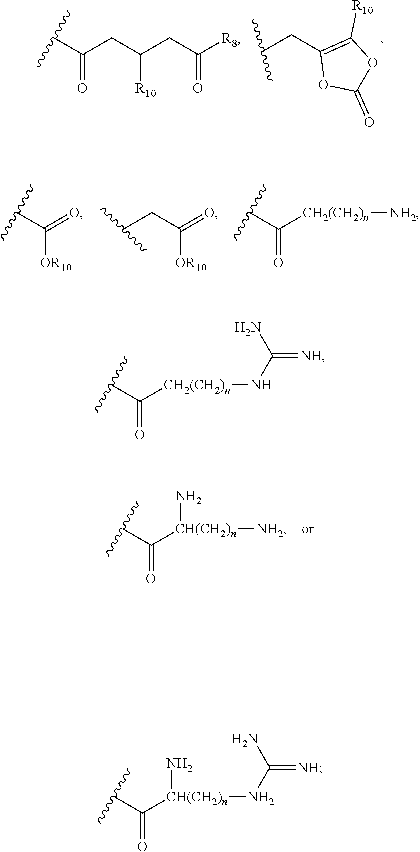

wherein R₁₀ and R₈ are alkyl; and
wherein n is an integer from 2 to 5.

In embodiments of Formula (I), R₁ and R₂ are independently C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; R₃ and R₄ are independently C₁-C₆ alkyl, C₁-C₆ heteroalkyl, C₁-C₆ haloalkyl, O—C₁-C₆ alkyl, O—C₁-C₆ heteroalkyl, O—C₁-C₆ haloalkyl; or R₃ and R₄ together with the carbon atom to which they are attached form a C₃-C₇ cycloalkyl; R₅ is C₁-C₆ alkyl, C₁-C₆ heteroalkyl, C₁-C₆ haloalkyl, O—C₁-C₆ alkyl, O—C₁-C₆ heteroalkyl, O—C₁-C₆ haloalkyl; and R₆ and R₇ are independently C₁-C₆ alkyl, C₁-C₆ allyl, C₁-C₆ haloalkyl, C₃-C₇ cycloalkyl, or C₃-C₇ heterocyclyl; or one of R₆ or R₇ is

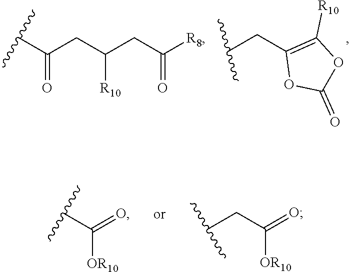

wherein R₁₀ and R₈ are independently C₁-C₆ alkyl or hydrogen.

In embodiments of Formula (I), X is F.

In embodiments of Formula (I), R₁ and R₂ are hydrogen.

In embodiments of Formula (I), R₃ and R₄ are hydrogen.

In embodiments of Formula (I), R₅ is C₁-C₆ alkyl. In embodiments, R₅ is methyl. In embodiments, R₅ is ethyl.

In embodiments of Formula (I), R₅ is hydrogen.

In embodiments of Formula (I), R₆ is C₁-C₆ alkyl and R₇ is hydrogen. In embodiments, R₆ is methyl. In embodiments, R₆ is ethyl.

In embodiments of Formula (I), R₆ and R₇ are hydrogen.

In embodiments of Formula (I), R₃ and R₄ together are carbonyl.

In embodiments of Formula (I), R₃ is OH.

In embodiments of Formula (I), R₃ and R₄ are deuterium.

In embodiments of Formula (I), R₃ is halogen. In some further embodiments, R₃ is F.

In embodiments of Formula (I), R₄ is halogen. In embodiments, R₄ is F.

In embodiments of Formula (I) R₆ is heterocyclyl. In embodiments, R₆ is

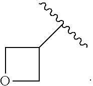 [SMILES: *C1COC1]

In embodiments, R₆ is

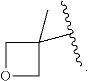 [SMILES: *C1(C)COC1]

In embodiments, R₆ is

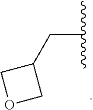 [SMILES: *CC1COC1]

In embodiments, R₆ is

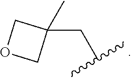 [SMILES: *CC1(C)COC1]

In embodiments of Formula (I), R₆ is

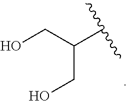 [SMILES: *C(CO)CO]

In embodiments, R₆ is

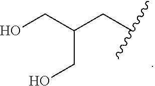 [SMILES: *CC(CO)CO]

In embodiments, R₆ is

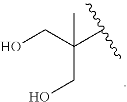 [SMILES: *C(C)(CO)CO]

In embodiments, R₆ is

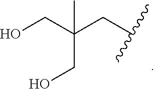 [SMILES: *CC(C)(CO)CO]

In embodiments, R₆ is

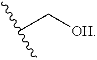 [SMILES: *CO]

In embodiments, R₆ is

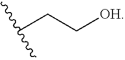 [SMILES: *CCO]

In embodiments, R₆ is

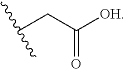 [SMILES: *CC(=O)O]

In embodiments, R₆ is

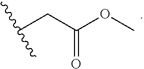 [SMILES: *CC(=O)OC]

In embodiments, R₆ is

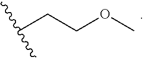 [SMILES: *CCOC]

In embodiments, the disclosure provides a compound of Formula (II):

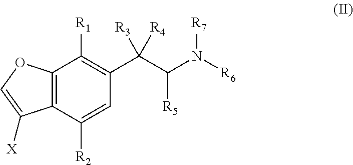

or a pharmaceutically acceptable salt thereof, wherein
X is halogen, deuterium, OH, OR₉, or CF₃;
   wherein R₉ is hydrogen, deuterium, halogen, alkyl, heteroalkyl, haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
   R₃ and R₄ together are carbonyl; or
   R₃ and R₄ together with the carbon atom to which they are attached form a cycloalkyl;
R₅ is hydrogen, deuterium, halogen, alkyl, deuterated alkyl, heteroalkyl, haloalkyl, O-alkyl, O-heteroalkyl, or O-haloalkyl; and
R₆ and R₇ are independently hydrogen, deuterium, alkyl, allyl, haloalkyl, cycloalkyl, or heterocyclyl, wherein the alkyl, deuterated alkyl, haloalkyl, cycloalkyl, or heterocyclyl are independently optionally substituted; or
   one of R₆ or R₇ is

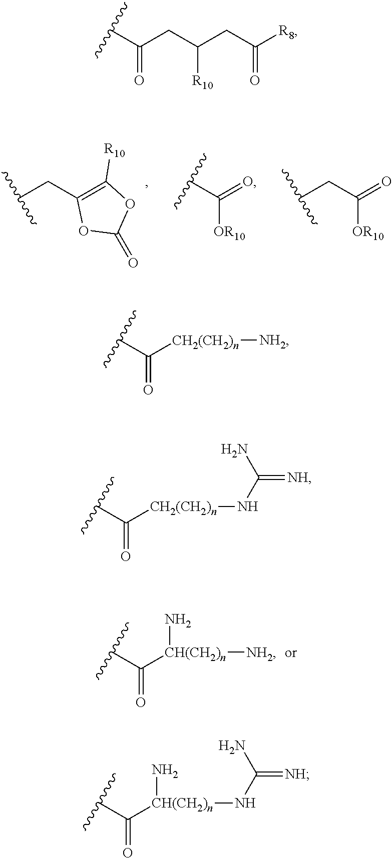

wherein R₁₀ and R₈ are alkyl or hydrogen; and
wherein n is an integer from 2 to 5.

In embodiments of Formula (II), R₁ and R₂ are independently C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; R₃ and R₄ are independently C₁-C₆ alkyl, C₁-C₆ heteroalkyl, C₁-C₆ haloalkyl, O—C₁-C₆ alkyl, O—C₁-C₆ heteroalkyl, O—C₁-C₆ haloalkyl; or R₃ and R₄ together with the carbon atom to which they are attached form a C₃-C₇ cycloalkyl; R₅ is C₁-C₆ alkyl, C₁-C₆ heteroalkyl, C₁-C₆ haloalkyl, O-alkyl, O-heteroalkyl, O-haloalkyl; or R₆ and R₇ are independently C₁-C₆ alkyl, C₁-C₆ allyl, C₁-C₆ haloalkyl, C₃-C₇ cycloalkyl, or C₃-C₇ heterocyclyl; or one of R₆ or R₇ is

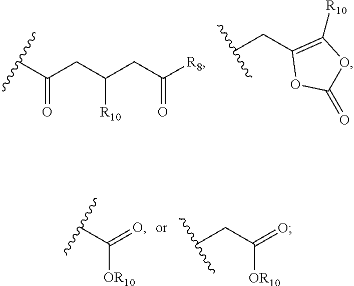

wherein R₁₀ and R₈ are independently C₁-C₆ alkyl or hydrogen.

In embodiments of Formula (II), X is F.

In embodiments of Formula (II), R₁ and R₂ are hydrogen.

In embodiments of Formula (II), R₅ is C₁-C₆ alkyl. In embodiments, R₅ is methyl. In embodiments, R₅ is ethyl.

In embodiments of Formula (II), R₆ is C₁-C₆ alkyl and R₇ is hydrogen. In embodiments, R₆ is methyl. In embodiments, R₆ is ethyl.

In embodiments of Formula (II), R₆ and R₇ are hydrogen.

In embodiments of Formula (II), R₃ and R₄ are hydrogen.

In embodiments of Formula (II), R₃ and R₄ together are carbonyl.

In embodiments of Formula (II), R₃ is OH.

In embodiments of Formula (II), R₃ and R₄ are deuterium.

In embodiments of Formula (II), R₃ is halogen. In embodiments, R₃ is F.

In embodiments of Formula (II), R₄ is halogen. In embodiments, R₄ is F.

In embodiments of Formula (II), R₆ is heterocyclyl. In embodiments, R₆ is

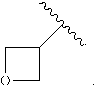 [SMILES: *C1COC1]

In embodiments, R₆ is

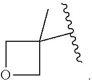 [SMILES: *C1(C)COC1]

In embodiments, R₆ is

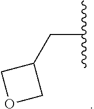 [SMILES: *CC1COC1]

In embodiments, R₆ is

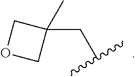 [SMILES: *CC1(C)COC1]

In embodiments of Formula (II), R₆ is

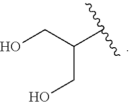 [SMILES: *C(CO)CO]

In embodiments, R₆ is

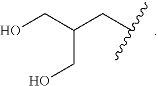 [SMILES: *CC(CO)CO]

In embodiments, R₆ is

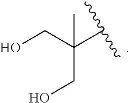 [SMILES: *C(C)(CO)CO]

In embodiments, R₆ is

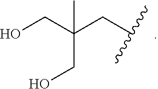 [SMILES: *CC(C)(CO)CO]

In embodiments, R₆ is

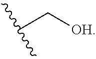 [SMILES: *CO]

In embodiments, R₆ is

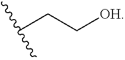 [SMILES: *CCO]

In embodiments, R₆ is

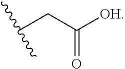 [SMILES: *CC(=O)O]

In embodiments, R₆ is

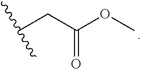 [SMILES: *CC(=O)OC]

In embodiments, R₆ is

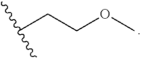 [SMILES: *CCOC]

In embodiments of Formula (I), the compound is a compound of Formula (I-B):

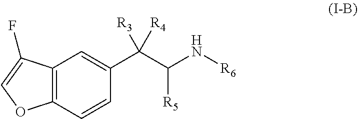

or a pharmaceutically acceptable salt thereof,
wherein,
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

In embodiments of Formula (II), the compound is a compound of Formula (II-B):

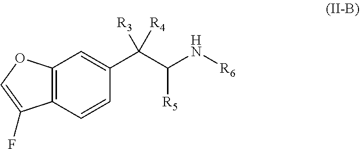

or a pharmaceutically acceptable salt thereof,
wherein,
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

In embodiments, the disclosure provides a composition comprising any of the compounds of the above-described aspects and embodiments, and a pharmaceutically acceptable carrier.

In embodiments, the disclosure provides a method for treating a neurological disorder, comprising administering to a subject in need thereof an effective amount of the compound or pharmaceutical composition of any of the above described aspects and embodiments. In embodiments, the neurological disorder is post-traumatic stress disorder. In embodiments, the neurological disorder is an eating disorder including anorexia nervosa or bulimia nervosa. In embodiments, the neurological disorder is obsessive-compulsive disorder (OCD).

Additional aspects and embodiments in accordance with the disclosure will be apparent to one of skill in the art in light of the following description.

### DETAILED DESCRIPTION

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### Definitions

The following terms and expressions used herein have the indicated meanings.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Cycloalkyl” refers to a stable non aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7 dimethyl bicyclo[2.2.1]heptanyl, and the like. Unless otherwise stated specifically in the specification, a cycloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, and 1,1 dioxo thiomorpholinyl. Unless stated otherwise specifically in the specification, a heterocyclyl group can be optionally substituted.

The term “thia” as used herein means a ═S group.

The term “saturated” as used herein means the referenced chemical structure does not contain any multiple carbon-carbon bonds. For example, a saturated cycloalkyl group as defined herein includes cyclohexyl, cyclopropyl, and the like.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

The term “substituted” used herein means any of the groups described herein (e.g., alkyl, alkenyl, alkynyl, alkoxy, aryl, cycloalkyl, cycloalkenyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with NR_{g}Rₕ, NR_{g}C(═O)Rₕ, NR_{g}C(═O)NR_{g}Rₕ, NR_{g}C(═O)ORₕ, NR_{g}SO₂Rₕ, OC(═O)NR_{g}Rₕ, OR_{g}, SR_{g}, SOR_{g}, SO₂R_{g}, OSO₂R_{g}, SO₂OR_{g}, ═NSO₂R_{g}, and SO₂NR_{g}Rₕ. “Substituted” also means any of the above groups in which one or more hydrogen atoms are replaced with C(═O)R_{g}, C(═O)OR_{g}, C(═O)NR_{g}Rₕ, CH₂SO₂R_{g}, CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. Treating can be improving, or at least partially ameliorating a disorder or an associated symptom of a disorder.

The term “preventing” as used herein with regard to a patient or subject, refers to preventing the onset of disease development if none had occurred, preventing the disease or disorder from occurring in a subject or a patient that may be predisposed to the disorder or disease but has not yet been diagnosed as having the disorder or disease, and/or preventing further disease/disorder development if already present.

The term “therapeutically effective” applied to dose or amount refers to that quantity of a compound or pharmaceutical formulation that is sufficient to result in a desired clinical benefit after administration to a patient in need thereof.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

### Compounds

In embodiments, the disclosure provides a compound of Formula (I):

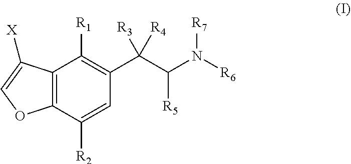

or a pharmaceutically acceptable salt thereof,
wherein,
X is halogen, deuterium, OH, OR₉, or CF₃;
   wherein R₉ is hydrogen, deuterium, halogen, alkyl, heteroalkyl, haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
   R₃ and R₄ together are carbonyl; or
   R₃ and R₄ together with the carbon atom to which they are attached form a cycloalkyl;
R₅ is hydrogen, deuterium, halogen, alkyl, deuterated alkyl, heteroalkyl, haloalkyl, O-alkyl, O-heteroalkyl, or O-haloalkyl; and
R₆ and R₇ are independently hydrogen, deuterium, alkyl, allyl, haloalkyl, cycloalkyl, or heterocyclyl, wherein the alkyl, deuterated alkyl, haloalkyl, cycloalkyl, or heterocyclyl are independently optionally substituted; or
   one of R₆ or R₇ is

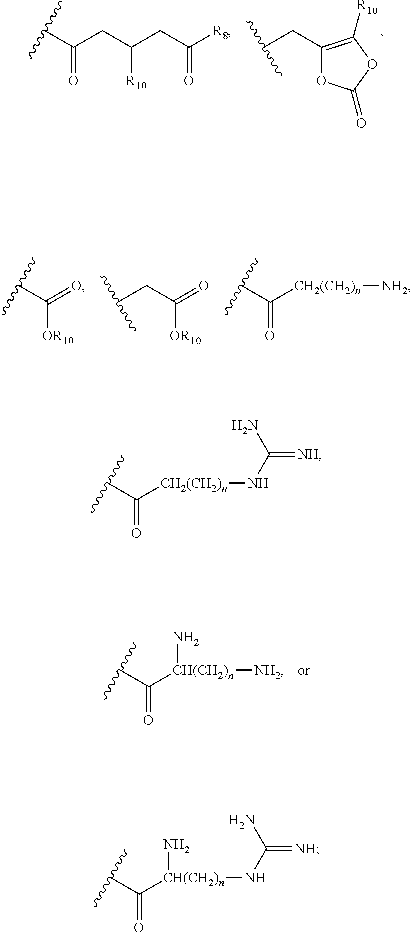

wherein R₁₀ and R₈ are alkyl or hydrogen; and
wherein n is an integer from 2 to 5.

In embodiments, the compound of Formula (I) is:

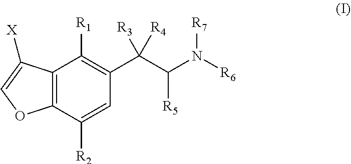

or a pharmaceutically acceptable salt thereof,
wherein,
X is halogen, deuterium, OH, OR₉, or CF₃;
   wherein R₉ is hydrogen, deuterium, halogen, C₁-C₆ alkyl, C₁-C₆ heteroalkyl, C₁-C₆ haloalkyl, aryl, or benzyl;
R₁ and R₂ are independently hydrogen, deuterium, halogen, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl;
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ and R₇ are independently hydrogen, deuterium, C₁-C₆ alkyl, C₁-C₆ allyl, C₁-C₆ heteroalkyl, C₁-C₆ haloalkyl, wherein the alkyl, deuterated alkyl, haloalkyl, cycloalkyl, or heterocyclyl are independently optionally substituted; or
   one of R₆ or R₇ is

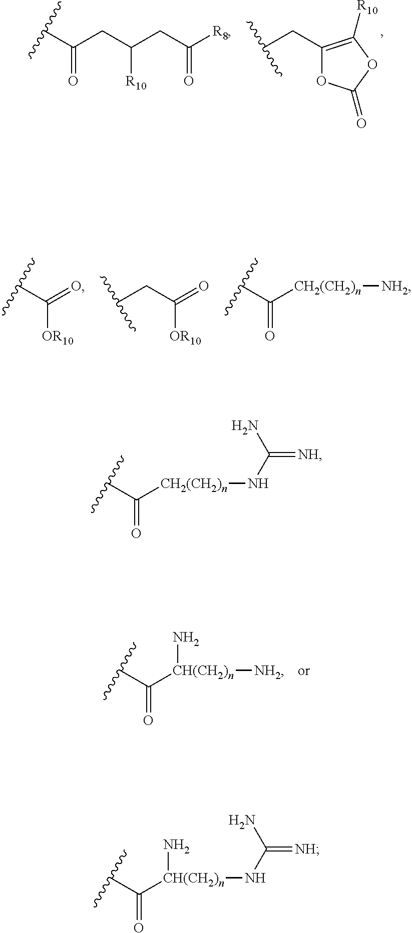

Wherein R₁₀ and R₈ are C₁-C₆ alkyl or hydrogen; and
wherein n is an integer from 2 to 5.

In embodiments of Formula (I), the compound is a compound of Formula (I-A):

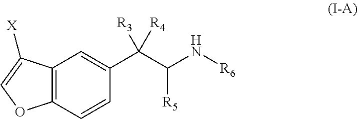

or a pharmaceutically acceptable salt thereof,
wherein,
X is halogen;
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

In embodiments, the compound of Formula (I) is a compound of Formula (I-B):

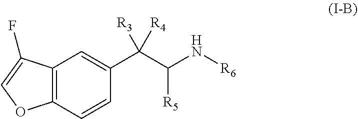

or a pharmaceutically acceptable salt thereof,
wherein
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

In embodiments, the disclosure provides a compound of Formula (II):

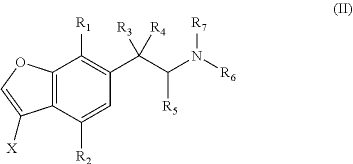

or a pharmaceutically acceptable salt thereof,
wherein,
X is halogen, deuterium, OH, OR₉, or CF₃;
   wherein R₉ is hydrogen, deuterium, halogen, alkyl, heteroalkyl, haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
   R₃ and R₄ together are carbonyl; or
   R₃ and R₄ together with the carbon atom to which they are attached form a cycloalkyl;
R₅ is hydrogen, deuterium, halogen, alkyl, deuterated alkyl, heteroalkyl, haloalkyl, O-alkyl, O-heteroalkyl, or O-haloalkyl; and
R₆ and R₇ are independently hydrogen, deuterium, alkyl, allyl, haloalkyl, cycloalkyl, or heterocyclyl, wherein the alkyl, deuterated alkyl, haloalkyl, cycloalkyl, or heterocyclyl are independently optionally substituted; or
   one of R₆ or R₇ is

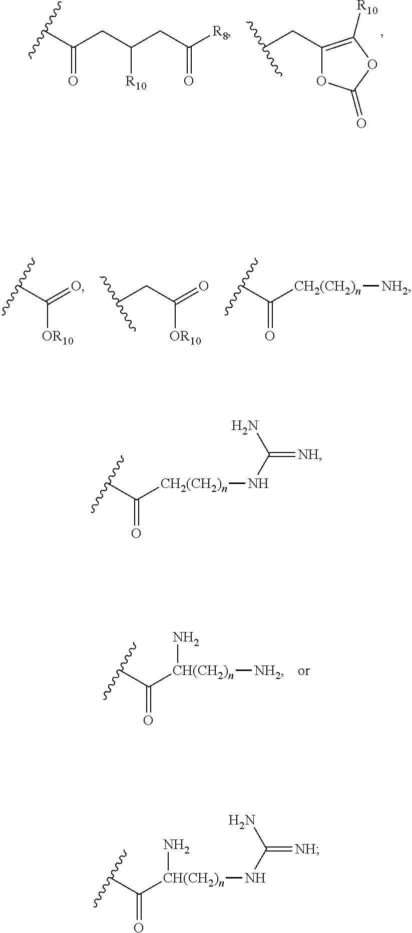

wherein R₁₀ and R₈ are alkyl or hydrogen; and
wherein n is an integer from 2 to 5.

In embodiments, the compound of Formula (II) is:

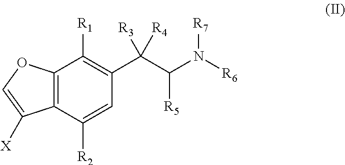

or a pharmaceutically acceptable salt thereof,
wherein,
X is halogen, deuterium, OH, OR₉, or CF₃;
   wherein R₉ is hydrogen, deuterium, halogen, C₁-C₆ alkyl, C₁-C₆ heteroalkyl, C₁-C₆ haloalkyl, aryl, or benzyl;
R₁ and R₂ are independently hydrogen, deuterium, halogen, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl;
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ and R₇ are independently hydrogen, deuterium, C₁-C₆ alkyl, C₁-C₆ heteroalkyl, C₁-C₆ haloalkyl, wherein the alkyl, deuterated alkyl, haloalkyl, cycloalkyl, or heterocyclyl are independently optionally substituted; or
   one of R₆ or R₇ is

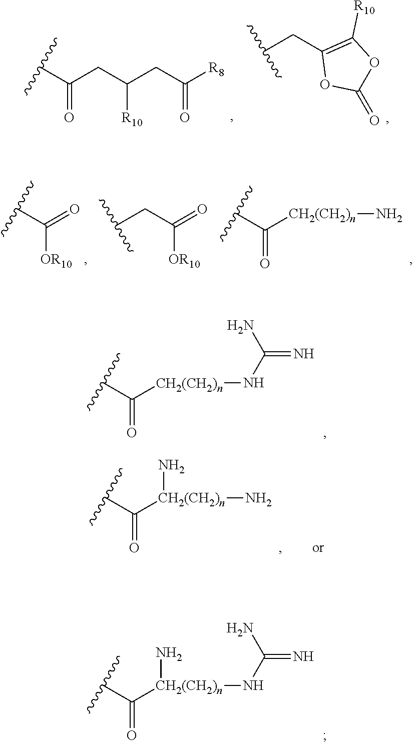

Wherein R₁₀ and R₈ are C₁-C₆ alkyl or hydrogen; and
wherein n is an integer from 2 to 5.

In embodiments, the compound of Formula (II) is a compound of Formula (II-A):

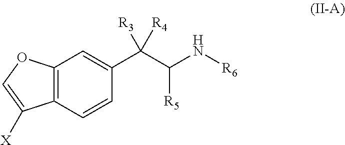

or a pharmaceutically acceptable salt thereof,
wherein
X is halogen;
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

In embodiments, the compound of Formula (I) is a compound of Formula (II-B):

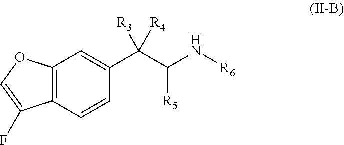

or a pharmaceutically acceptable salt thereof,
wherein,
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

In embodiments of the compounds of Formula (I) or (II), R₁ and R₂ are independently hydrogen, deuterium, halogen, R₁ and R₂ are independently C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl.

In embodiments of the compounds of Formula (I), (I-A), (I-B), (II), (II-A), or (II-B), R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl.

In embodiments of the compounds of Formula (I), (I-A), (I-B), (II), (II-A), or (II-B), R₃ and R₄ together are carbonyl.

In embodiments of the compounds of Formula (I), (I-A), (I-B), (II), (II-A), or (II-B), R₃ and R₄ together with the carbon atom to which they are attached form a C₃-C₇ cycloalkyl.

In embodiments of the compounds of Formula (I), (I-A), (I-B), (II), (II-A), or (II-B), R₅ is hydrogen, deuterium, halogen, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl.

In embodiments of the compounds of Formula (I), (I-A), (I-B), (II), (II-A), or (II-B), R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl.

In embodiments of the compounds of Formula (I), (I-A), (I-B), (II), (II-A), or (II-B), R₆ and R₇ are independently hydrogen, deuterium, C₁-C₆ alkyl, C₁-C₆ allyl, C₁-C₆ haloalkyl, C₃-C₇ cycloalkyl, or C₃-C₇ heterocyclyl.

In embodiments of the compounds of Formula (I), (I-A), (I-B), (II), (II-A), or (II-B), R₆ and R₇ are independently hydrogen, deuterium, C₁-C₆ alkyl, C₁-C₆ allyl, C₁-C₆ haloalkyl, C₃-C₇ cycloalkyl, or C₃-C₇ heterocyclyl, wherein the alkyl, haloalkyl, cycloalkyl, or heterocyclyl are independently optionally substituted.

In embodiments of the compounds of Formula (I) or (II), X is F.

In embodiments of the compounds of Formula (I) or (II), R₁ and R₂ are hydrogen.

In embodiments of the compounds of Formula (I), (I-A), (I-B), (II), (II-A), or (II-B), R₃ and R₄ are hydrogen.

In embodiments of the compounds of Formula (I), (I-A), (I-B), (II), (II-A), or (II-B), R₃ and R₄ are deuterium.

In embodiments of the compounds of Formula (I), (I-A), (I-B), (II), (II-A), or (II-B), R₅ is hydrogen.

In embodiments of the compounds of Formula (I), (I-A), (I-B), (II), (II-A), or (II-B), R₅ is C₁-C₆ alkyl. In embodiments, R₅ is methyl. In embodiments, R₅ is ethyl.

In embodiments of the compounds of Formula (I) or (II), R₆ and R₇ are hydrogen.

In embodiments of the compounds of Formula (I) or (II), R₆ is C₁-C₆ alkyl and R₇ is hydrogen. In embodiments, R₆ is methyl. In embodiments, R₆ is ethyl.

In embodiments of the compounds of Formula (I), (I-A), (I-B), (II), (II-A), or (II-B), R₃ and R₄ together are carbonyl.

In embodiments of the compounds of Formula (I), (I-A), (I-B), (II), (II-A), or (II-B), R₃ is OH.

In embodiments of the compounds of Formula (I), (I-A), (I-B), (II), (II-A), or (II-B), R₃ is halogen. In embodiments, R₃ is F.

In embodiments of the compounds of Formula (I), (I-A), (I-B), (II), (II-A), or (II-B), R₄ is halogen. In embodiments, R₄ is F.

In embodiments of the compounds of Formula (I), (I-A), (I-B), (II), (II-A), or (II-B), R₆ is heterocycyl. In embodiments, R₆ is

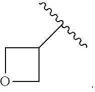 [SMILES: *C1COC1]

In embodiments, R₆ is

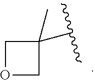 [SMILES: *C1(C)COC1]

In embodiments, R₆ is

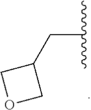 [SMILES: *CC1COC1]

In embodiments, R₆ is

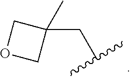 [SMILES: *CC1(C)COC1]

In embodiments of the compounds of Formula (I), (I-A), (I-B), (II), (II-A), or (II-B), R₆ is C₁-C₆ alkyl substituted with one or more functional groups. For example, in embodiments, R₆ is

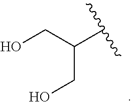 [SMILES: *C(CO)CO]

In embodiments, R₆ is

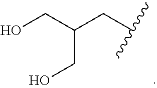 [SMILES: *CC(CO)CO]

In embodiments, R₆ is

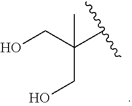 [SMILES: *C(C)(CO)CO]

In embodiments, R₆ is

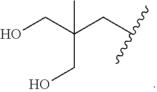 [SMILES: *CC(C)(CO)CO]

In embodiments, R₆ is

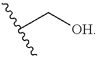 [SMILES: *CO]

In embodiments, R₆ is

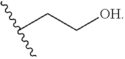 [SMILES: *CCO]

In embodiments, R₆ is

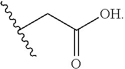 [SMILES: *CC(=O)O]

In embodiments, R₆ is

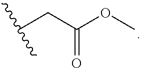 [SMILES: *CC(=O)OC]

In embodiments, R₆ is

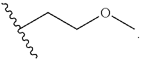 [SMILES: *CCOC]

In embodiments, the compounds are selected from the following group in Table 1.

TABLE 1
Illustrative Compounds

| Compound | Structure | Name |
|---|---|---|
| 1 | 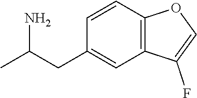 | 1-(3-fluorobenzofuran-5- yl)propan-2-amine |
| 2 | 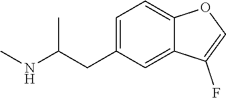 | 1-(3-fluorobenzofuran-5-yl)-N- methylpropan-2-amine |
| 3 | 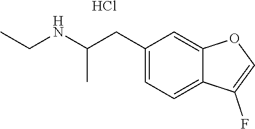 [SMILES: Cl ; CCNC(C)Cc1ccc2c(F)coc2c1] | N-ethyl-1-(3-fluorobenzofuran-6- yl)propan-2-amine hydrochloride |
| 4 | 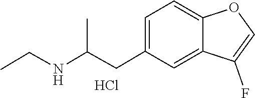 [SMILES: CCNC(C)Cc1ccc2occ(F)c2c1 ; Cl] | N-ethyl-1-(3-fluorobenzofuran-5- yl)propan-2-amine hydrochloride |
| 5 | 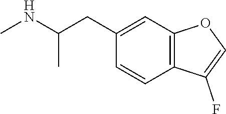 | 1-(3-fluorobenzofuran-6-yl)-N- methylpropan-2-amine |
| 6 | 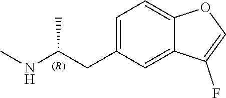 | (R)-1-(3-fluorobenzofuran-5-yl)- N-methylpropan-2-amine |
| 7 | 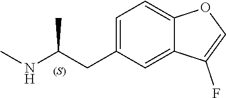 | (S)-1-(3-fluorobenzofuran-5-yl)- N-methylpropan-2-amine |
| 8 | 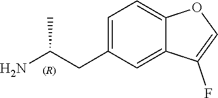 | (R)-1-(3-fluorobenzofuran-5- yl)propan-2-amine |
| 9 | 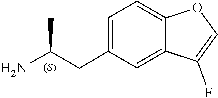 | (S)-1-(3-fluorobenzofuran-5- yl)propan-2-amine |
| 10 | 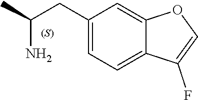 | (S)-1-(3-fluorobenzofuran-6- yl)propan-2-amine |
| 11 | 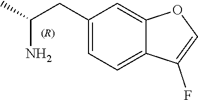 | (R)-1-(3-fluorobenzofuran-6- yl)propan-2-amine |
| 12 | 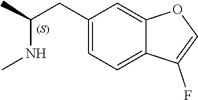 | (S)-1-(3-fluorobenzofuran-6-yl)- N-methylpropan-2-amine |
| 13 | 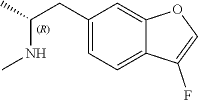 | (R)-1-(3-fluorobenzofuran-6-yl)- N-methylpropan-2-amine |
| 14 | 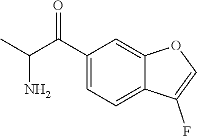 | 2-amino-1-(3-fluorobenzofuran- 6-yl)propan-1-one |
| 15 | 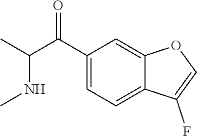 [SMILES: CNC(C)C(=O)c1ccc2c(F)coc2c1] | 1-(3-fluorobenzofuran-6-yl)-2- (methylamino)propan-1-one |
| 16 | 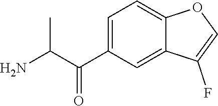 | 2-amino-1-(3-fluorobenzofuran- 5-yl)propan-1-one |
| 17 | 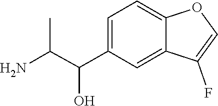 | 2-amino-1-(3-fluorobenzofuran- 5-yl)propan-1-ol |
| 18 | 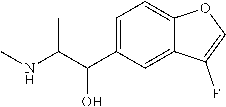 [SMILES: CNC(C)C(O)c1ccc2occ(F)c2c1] | 1-(3-fluorobenzofuran-5-yl)-2- (methylamino)propan-1-ol |
| 19 | 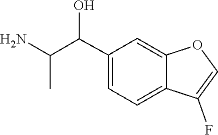 | 2-amino-1-(3-fluorobenzofuran- 6-yl)propan-1-ol |
| 20 | 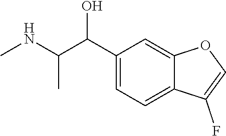 [SMILES: CNC(C)C(O)c1ccc2c(F)coc2c1] | 1-(3-fluorobenzofuran-6-yl)-2- (methylamino)propan-1-ol |
| 21 | 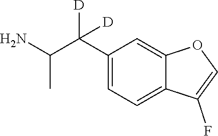 [SMILES: [2H]C([2H])(c1ccc2c(F)coc2c1)C(C)N] | 1-(3-fluorobenzofuran-6- yl)propan-1,1-d₂-2-amine |
| 22 | 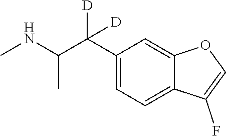 [SMILES: [2H]C([2H])(c1ccc2c(F)coc2c1)C(C)NC] | 1-(3-fluorobenzofuran-6-yl)-N- methylpropan-1,1-d₂-2-amine |
| 23 | 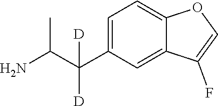 [SMILES: [2H]C([2H])(c1ccc2occ(F)c2c1)C(C)N] | 1-(3-fluorobenzofuran-5- yl)propan-1,1-d₂-2-amine |
| 24 | 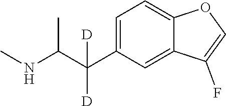 [SMILES: [2H]C([2H])(c1ccc2occ(F)c2c1)C(C)NC] | 1-(3-fluorobenzofuran-5-yl)-N- methylpropan-1,1-d₂-2-amine |
| 25 | 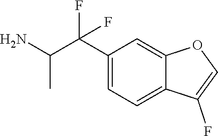 [SMILES: CC(N)C(F)(F)c1ccc2c(F)coc2c1] | 1,1-difluoro-1-(3- fluorobenzofuran-6-yl)propan-2- amine |
| 26 | 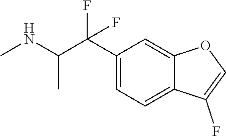 [SMILES: CNC(C)C(F)(F)c1ccc2c(F)coc2c1] | 1,1-difluoro-1-(3- fluorobenzofuran-6-yl)-N- methylpropan-2-amine |
| 27 | 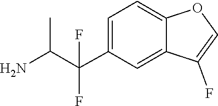 [SMILES: CC(N)C(F)(F)c1ccc2occ(F)c2c1] | 1,1-difluoro-1-(3- fluorobenzofuran-5-yl)propan-2- amine |
| 28 | 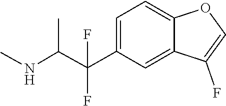 [SMILES: CNC(C)C(F)(F)c1ccc2occ(F)c2c1] | 1,1-difluoro-1-(3- fluorobenzofuran-5-yl)-N- methylpropan-2-amine |
| 29 | 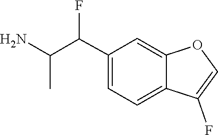 | 1-fluoro-1-(3-fluorobenzofuran-6- yl)propan-2-amine |
| 30 | 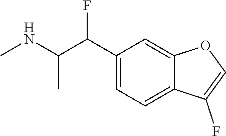 [SMILES: CNC(C)C(F)c1ccc2c(F)coc2c1] | 1-fluoro-1-(3-fluorobenzofuran-6- yl)-N-methylpropan-2-amine |
| 31 | 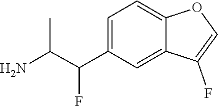 | 1-fluoro-1-(3-fluorobenzofuran-5- yl)propan-2-amine |
| 32 | 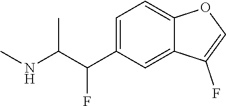 [SMILES: CNC(C)C(F)c1ccc2occ(F)c2c1] | 1-fluoro-1-(3-fluorobenzofuran-5- yl)-N-methylpropan-2-amine |
| 33 | 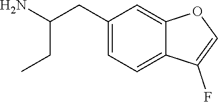 | 1-(3-fluorobenzofuran-6-yl)butan- 2-amine |
| 34 | 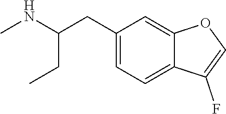 [SMILES: CCC(Cc1ccc2c(F)coc2c1)NC] | 1-(3-fluorobenzofuran-6-yl)-N- methylbutan-2-amine |
| 35 | 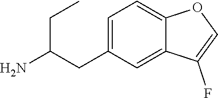 | 1-(3-fluorobenzofuran-5-yl)butan- 2-amine |
| 36 | 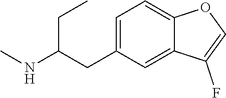 [SMILES: CCC(Cc1ccc2occ(F)c2c1)NC] | 1-(3-fluorobenzofuran-5-yl)-N- methylbutan-2-amine |
| 37 | 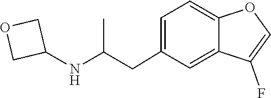 [SMILES: CC(Cc1ccc2occ(F)c2c1)NC1COC1] | N-(1-(3-fluorobenzofuran-5- yl)propan-2-yl)oxetan-3-amine |
| 38 | 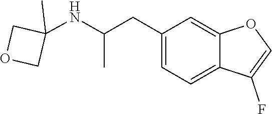 [SMILES: CC(Cc1ccc2c(F)coc2c1)NC1(C)COC1] | N-(1-(3-fluorobenzofuran-6- yl)propan-2-yl)-3-methyloxetan- 3-amine |
| 39 | 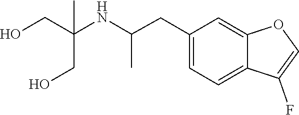 [SMILES: CC(Cc1ccc2c(F)coc2c1)NC(C)(CO)CO] | 2-((1-(3-fluorobenzofuran-6- yl)propan-2-yl)amino)-2- methylpropane-1,3-diol |
| 40 | 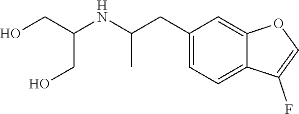 [SMILES: CC(Cc1ccc2c(F)coc2c1)NC(CO)CO] | 2-((1-(3-fluorobenzofuran-6- yl)propan-2-yl)amino)propane- 1,3-diol |
| 41 | 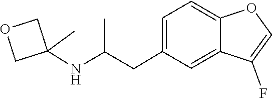 [SMILES: CC(Cc1ccc2occ(F)c2c1)NC1(C)COC1] | N-(1-(3-fluorobenzofuran-5- yl)propan-2-yl)-3-methyloxetan- 3-amine |
| 42 | 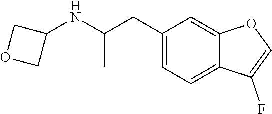 [SMILES: CC(Cc1ccc2c(F)coc2c1)NC1COC1] | N-(1-(3-fluorobenzofuran-6- yl)propan-2-yl)oxetan-3-amine |
| 43 | 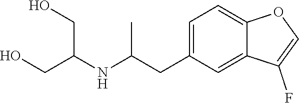 [SMILES: CC(Cc1ccc2occ(F)c2c1)NC(CO)CO] | 2-((1-(3-fluorobenzofuran-5- yl)propan-2-yl)amino)propane- 1,3-diol |
| 44 | 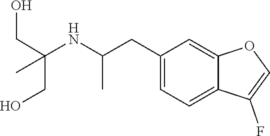 [SMILES: CC(Cc1ccc2c(F)coc2c1)NC(C)(CO)CO] | 2-((1-(3-fluorobenzofuran-6- yl)propan-2-yl)amino)-2- methylpropane-1,3-diol |
| 45 | 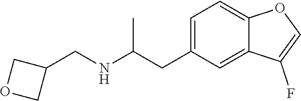 [SMILES: CC(Cc1ccc2occ(F)c2c1)NCC1COC1] | 1-(3-fluorobenzofuran-5-yl)-N- (oxetan-3-ylmethyl)propan-2- amine |
| 46 | 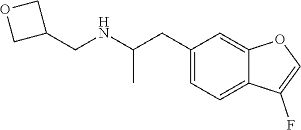 [SMILES: CC(Cc1ccc2c(F)coc2c1)NCC1COC1] | 1-(3-fluorobenzofuran-6-yl)-N- (oxetan-3-ylmethyl)propan-2- amine |
| 47 | 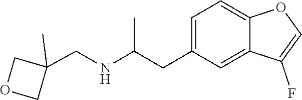 [SMILES: CC(Cc1ccc2occ(F)c2c1)NCC1(C)COC1] | 1-(3-fluorobenzofuran-5-yl)-N- ((3-methyloxetan-3- yl)methyl)propan-2-amine |
| 48 | 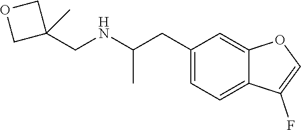 [SMILES: CC(Cc1ccc2c(F)coc2c1)NCC1(C)COC1] | 1-(3-fluorobenzofuran-6-yl)-N- ((3-methyloxetan-3- yl)methyl)propan-2-amine |
| 49 | 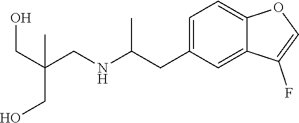 [SMILES: CC(Cc1ccc2occ(F)c2c1)NCC(C)(CO)CO] | 2-(((1-(3-fluorobenzofuran-5- yl)propan-2-yl)amino)methyl)-2- methylpropane-1,3-diol |
| 50 | 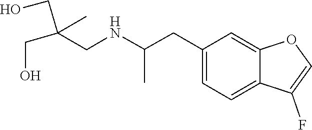 [SMILES: CC(Cc1ccc2c(F)coc2c1)NCC(C)(CO)CO] | 2-(((1-(3-fluorobenzofuran-6- yl)propan-2-yl)amino)methyl)-2- methylpropane-1,3-diol |
| 51 | 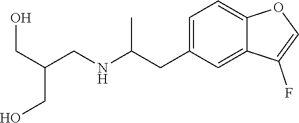 [SMILES: CC(Cc1ccc2occ(F)c2c1)NCC(CO)CO] | 2-(((1-(3-fluorobenzofuran-5- yl)propan-2- yl)amino)methyl)propane-1,3-diol |
| 52 | 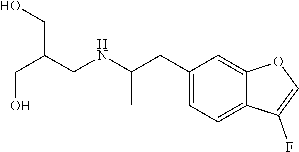 [SMILES: CC(Cc1ccc2c(F)coc2c1)NCC(CO)CO] | 2-(((1-(3-fluorobenzofuran-6- yl)propan-2- yl)amino)methyl)propane-1,3-diol |
| 53 | 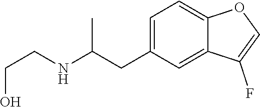 [SMILES: CC(Cc1ccc2occ(F)c2c1)NCCO] | 2-((1-(3-fluorobenzofuran-5- yl)propan-2-yl)amino)ethan-1-ol |
| 54 | 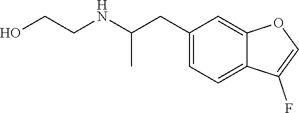 [SMILES: CC(Cc1ccc2c(F)coc2c1)NCCO] | 2-((1-(3-fluorobenzofuran-6- yl)propan-2-yl)amino)ethan-1-ol |
| 55 | 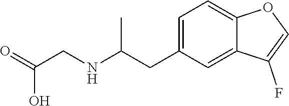 [SMILES: CC(Cc1ccc2occ(F)c2c1)NCC(=O)O] | (1-(3-fluorobenzofuran-5- yl)propan-2-yl)glycine |
| 56 | 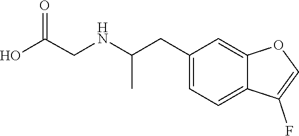 [SMILES: CC(Cc1ccc2c(F)coc2c1)NCC(=O)O] | (1-(3-fluorobenzofuran-6- yl)propan-2-yl)glycine |
| 57 | 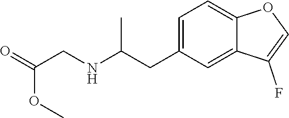 [SMILES: COC(=O)CNC(C)Cc1ccc2occ(F)c2c1] | methyl (1-(3-fluorobenzofuran-5- yl)propan-2-yl)glycinate |
| 58 | 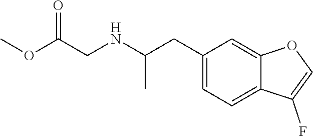 [SMILES: COC(=O)CNC(C)Cc1ccc2c(F)coc2c1] | methyl (1-(3-fluorobenzofuran-6- yl)propan-2-yl)glycinate |
| 59 | 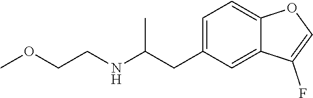 [SMILES: COCCNC(C)Cc1ccc2occ(F)c2c1] | 1-(3-fluorobenzofuran-5-yl)-N-(2- methoxyethyl)propan-2-amine |
| 60 | 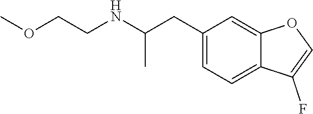 [SMILES: COCCNC(C)Cc1ccc2c(F)coc2c1] | 1-(3-fluorobenzofuran-6-yl)-N-(2- methoxyethyl)propan-2-amine |
| 61 | 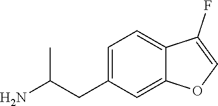 | 1-(3-fluorobenzofuran-6- yl)propan-2-amine |
| 62 | 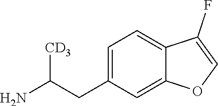 [SMILES: [2H]C([2H])([2H])C(N)Cc1ccc2c(F)coc2c1] | 1-(3-fluorobenzofuran-6- yl)propan-3,3,3,-d₂-2-amine |
| 63 | 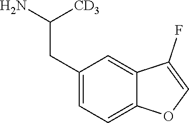 [SMILES: [2H]C([2H])([2H])C(N)Cc1ccc2occ(F)c2c1] | 1-(3-fluorobenzofuran-5- yl)propan-3,3,3-d₂-2-amine |
| 64 | 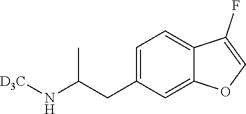 [SMILES: [2H]C([2H])([2H])NC(C)Cc1ccc2c(F)coc2c1] | 1-(3-fluorobenzofuran-6-yl)-N- trideuteromethylpropan-2-amine |
| 65 | 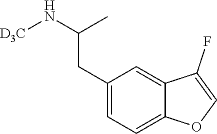 [SMILES: [2H]C([2H])([2H])NC(C)Cc1ccc2occ(F)c2c1] | 1-(3-fluorobenzofuran-5-yl)-N- trideuteromethylpropan-2-amine |

### Methods, Uses, and Therapeutics Applications

In embodiments, the present disclosure provides a method of treating or preventing neurological disorders in a subject in need thereof, the method comprising administering to the subject a therapeutically effective amount of a compound disclosed herein, e.g., a compound of Formula (I), (I-A), (I-B), (II), (II-A), or (II-B), or Table 1, or a pharmaceutically acceptable salt thereof.

As referred to herein, a “neurological disease” refers to any condition, disorder, or disease involving the nervous system, for example, diseases that involve the central nervous system (brain, brainstem and cerebellum), the peripheral nervous system (including cranial nerves), and the autonomic nervous system.

Within the broad scope of neurological diseases, a “neurodegenerative disease” refers to a neurological disease marked by the loss of nerve cells or damage to nerve cells, including non-limiting examples of Alzheimer's disease, Lewy body dementia, Parkinson's disease, amyotrophic lateral sclerosis, tauopathies (including frontotemporal dementia), Huntington's disease, and the like. Further non-limiting examples of neurological diseases include headache, stupor and coma, dementia, seizure, sleep disorders, trauma, infections, neoplasms, neuro-ophthalmological conditions, movement disorders, demyelinating diseases, spinal cord disorders, disorders of peripheral nerves, muscle and neuromuscular junctions, among others. In embodiments, addiction, mental illness, psychiatric disorders, and personality disorders are also included within the scope of neurological disorders and diseases, and thus include a broad scope of conditions such as those discussed herein and as generally known in the art.

The term “psychiatric disorder” refers to a condition, disorder, or disease of the mind and includes diseases and disorders listed in the Diagnostic and Statistical Manual of Mental Disorders—Fifth Edition (DSM-V), published by the American Psychiatric Association, Washington D.C. Psychiatric disorders include anxiety disorders (e.g., acute stress disorder agoraphobia, generalized anxiety disorder, obsessive-compulsive disorder, panic disorder, post-traumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder, among others.

In embodiments, the neurological disorder is a mood disorder. In embodiments, the mood disorder is clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, major depressive disorder, minor depressive disorder, bipolar disorder or manic depressive disorder, depression caused by chronic medical conditions, treatment-resistant depression, refractory depression, suicidality, suicidal ideation, or suicidal behavior. In embodiments, the method described herein provides therapeutic effect to a subject suffering from depression (e.g., moderate or severe depression). In embodiments, the mood disorder is associated with neuroendocrine diseases and disorders, neurodegenerative diseases and disorders (e.g., epilepsy), movement disorders, tremor (e.g., Parkinson's Disease), or women's health disorders or conditions. In embodiments the mood disorder is depression. In embodiments, the mood disorder is treatment-resistant depression or major depressive disorder. In embodiments, the mood disorder is major depressive disorder. In embodiments, the mood disorder is treatment-resistant depression.

In embodiments, the present disclosure provides a method of treating or preventing post-traumatic stress disorder (PTSD), mood disorders, general anxiety disorder, addictive disorders, and/or drug dependence in a subject in need thereof, the method comprising administering to the subject a therapeutically effective amount of a compound disclosed herein, e.g., a compound of Formula (I), (I-A), (I-B), (II), (II-A), or (II-B), or Table 1, or a pharmaceutically acceptable salt there, or a pharmaceutical composition thereof.

In embodiments, the compounds of the present disclosure are used to treat PTSD. In embodiments, the compounds of the present disclosure are used for induction and maintenance therapy to treat PTSD. In embodiments, the compounds of the present disclosure are used to treat PTSD with an improved safety profile when compared to treatment with the entactogenic, oneirophrenic or psychedelic compound (e.g. MDMA or related compound, psilocybin or dimethyltryptamine) alone. In embodiments, the compounds of the present disclosure are used for induction and maintenance therapy to treat PTSD with an improved safety profile when compared to treatment with the entactogenic, oneirophrenic or psychedelic compound (e.g. MDMA or related compound, psilocybin or dimethyltryptamine) alone.

In embodiments, the compounds of the present disclosure are used to treat behavioral or mood disorders. Examples of behavioral or mood disorders include anxiety, such as social anxiety in autistic subjects (e.g. autistic adults) and anxiety related to life-threatening illnesses, stress (where moderation thereof is measured, for example, by effects on amygdala responses). In embodiments, the anxiety disorder is panic disorder, obsessive-compulsive disorder, or general anxiety disorder. Other examples include lack of motivation, attention, accuracy, speed of response, perseveration, and/or cognitive engagement. Further examples include depression (e.g., MDD or TRD), attention disorders, disorders of executive function and/or cognitive engagement, obsessive compulsive disorder, bipolar disorder, panic disorder, phobia, schizophrenia, psychopathy, antisocial personality disorder and/or neurocognitive disorders.

In embodiments, the compounds the present disclosure are used to treat an addictive disorder. In embodiments, the addictive disorder is alcohol abuse, substance abuse, smoking, or obesity. In embodiments, the disorder is an eating disorder (anorexia nervosa, bulimia nervosa, binge eating disorder, etc.) or an auditory disorder.

In embodiments, the compounds of the present disclosure are used to treat aggression or an aggressive behavior that is associated with one or more CNS (i.e., neurological) disorders or diseases. For example, in embodiments, the compounds of the present disclosure are used to treat aggression or an aggressive behavior that is associated with a psychiatric disorder.

In embodiments, the disorder is an impulsive disorder. In embodiments, the impulsive disorder is attention deficit hyperactivity disorder (ADHD), attention deficit disorder (ADD), Tourette's syndrome or autism.

In embodiments, the disorder is a compulsive disorder. In embodiments, the compulsive disorder is obsessive compulsive disorder (OCD), gambling, or aberrant sexual behavior.

In embodiments, the disorder is a personality disorder. In embodiments, the personality disorder is conduct disorder, antisocial personality, or aggressive behavior.

### Pharmaceutical Compositions

In embodiments of the present disclosure, a pharmaceutical composition comprising a therapeutically effective amount of one or more compounds of the present disclosure (e.g., a compound of Formula (I), (I-A), (I-B), (II), (II-A), or (II-B), or Table 1) or a pharmaceutically acceptable salt thereof and a pharmaceutically acceptable excipient is provided.

For administration as compositions, including pharmaceutical compositions, the compounds are ordinarily combined with one or more carriers, diluents, and/or adjuvants appropriate for the indicated route of administration. The compounds may be mixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds described herein may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compounds can be prepared in a solid form (including granules, powders or suppositories) or in a liquid form (e.g., solutions, suspensions, or emulsions). The disclosed compounds may be applied in a variety of solutions and may be subjected to conventional pharmaceutical operations such as sterilization and/or may contain conventional adjuvants, such as preservatives, stabilizers, wetting agents, emulsifiers, buffers, etc.

The disclosed compounds may be administered orally, topically, parenterally, by inhalation or spray or rectally in dosage unit formulations containing conventional non-toxic pharmaceutically acceptable carriers, adjuvants, and vehicles. The term parenteral as used herein includes percutaneous, subcutaneous, intravascular (e.g., intravenous), intramuscular, or intrathecal injection or infusion techniques and the like. One or more compounds in accordance with the disclosure may be present in association with one or more non-toxic pharmaceutically acceptable carriers and/or diluents and/or adjuvants, and if desired other active ingredients. Such pharmaceutical compositions may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions in accordance with the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds and pharmaceutical compositions in accordance with the disclosure may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds and pharmaceutical compositions in accordance with the disclosure may be administered parenterally in a sterile medium. The drug, depending on the vehicle and concentration used, can either be suspended or dissolved in the vehicle.

### Numbered Embodiments

1. A compound of Formula (I):

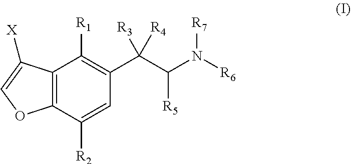

or a pharmaceutically acceptable salt thereof,
wherein,
X is halogen, deuterium, OH, OR₉, or CF₃;
   wherein R₉ is hydrogen, deuterium, halogen, alkyl, heteroalkyl, haloalkyl, O-alkyl, O-heteroalkyl, O-haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
   R₃ and R₄ together are carbonyl; or
   R₃ and R₄ together with the carbon atom to which they are attached form a cycloalkyl;
R₅ is hydrogen, deuterium, halogen, alkyl, heteroalkyl, haloalkyl, O-alkyl, O-heteroalkyl, or O-haloalkyl; and
R₆ and R₇ are independently hydrogen, deuterium, alkyl, haloalkyl, cycloalkyl, or heterocyclyl; or
   one of R₆ or R₇ is

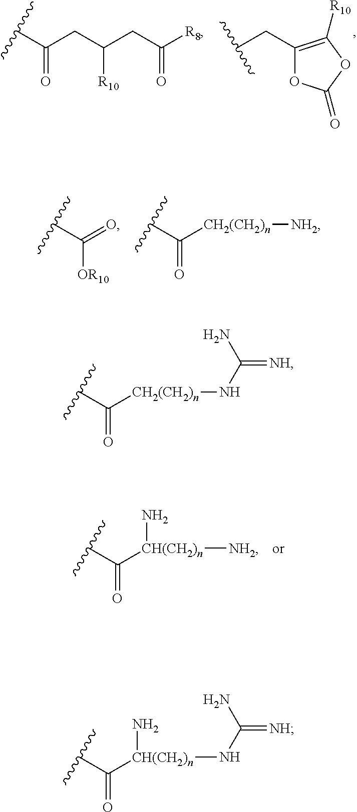

wherein R₁₀ and R₈ are alkyl; and
wherein n is an integer from 2 to 5.
2. The compound of embodiment 1, wherein
R₁ and R₂ are independently C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl;
R₃ and R₄ are independently C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together with the carbon atom to which they are attached form a C₃-C₇ cycloalkyl;
R₅ is C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ and R₇ are independently C₁-C₆ alkyl, C₁-C₆ haloalkyl, C₃-C₇ cycloalkyl, C₃-C₇ heterocyclyl; or
   one of R₆ or R₇ is

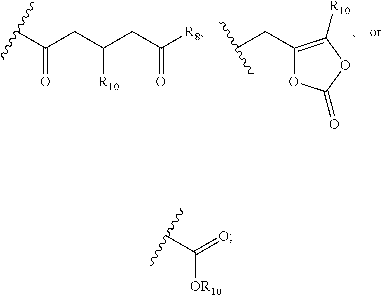

wherein R₁₀ and R₈ are independently C₁-C₆ alkyl.
3. The compound of any of embodiments 1-2, wherein X is F.
4. The compound of any of embodiments 1-3, wherein R₁ and R₂ are hydrogen.
5. The compound of any of embodiments 1-4, wherein R₃ and R₄ are hydrogen.
6. The compound of any of embodiments 1-5, wherein R₅ is C₁-C₆ alkyl.
7. The compound of embodiment 6, wherein R₅ is methyl.
8. The compound of embodiment 6, wherein R₅ is ethyl.
9. The compound of embodiment 6, wherein R₅ is hydrogen.
10. The compound of any of embodiments 1-9, wherein R₆ is C₁-C₆ alkyl and R₇ is hydrogen.
11. The compound of embodiment 10, wherein R₆ is methyl.
12. The compound of embodiment 10, wherein R₆ is ethyl.
13. The compound of any of claims 1-9, wherein R₆ and R₇ are hydrogen.
14. The compound of any of embodiments 1-13, wherein R₃ and R₄ together are carbonyl.
15. The compound of any of embodiments 1-13, wherein R₃ is OH.
16. The compound of any of embodiments 1-13, wherein R₃ and R₄ are deuterium.
17. The compound of any of embodiments 1-13, wherein R₃ is halogen.
18. The compound of embodiment 17, wherein R₃ is F.
19. The compound of embodiment 17, wherein R₄ is halogen.
20. The compound of embodiment 19, wherein R₄ is F.
21. The compound of any of embodiments 1-9 or 14-19 wherein R₆ is heterocyclyl.
22. The compound of embodiment 21, wherein R₆ is

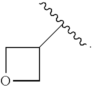 [SMILES: *C1COC1]

23. The compound of embodiment 21, wherein R₆ is

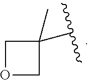 [SMILES: *C1(C)COC1]

24. The compound of embodiment 21, wherein R₆ is

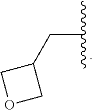 [SMILES: *CC1COC1]

25. The compound of embodiment 21, wherein R₆ is

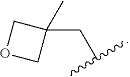 [SMILES: *CC1(C)COC1]

26. The compound of any of embodiments 1-9 or 14-20, wherein R₆ is

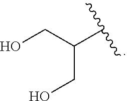 [SMILES: *C(CO)CO]

27. The compound of any of embodiments 1-9 or 14-20, wherein R₆ is

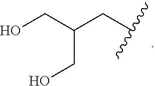 [SMILES: *CC(CO)CO]

28. The compound of any of embodiments 1-9 or 14-20, wherein R₆ is

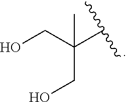 [SMILES: *C(C)(CO)CO]

29. The compound of any of embodiments 1-9 or 14-20, wherein R₆ is

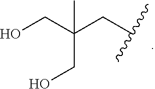 [SMILES: *CC(C)(CO)CO]

30. The compound of any of embodiments 1-9 or 14-20, wherein R₆ is

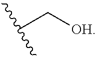 [SMILES: *CO]

31. The compound of any of embodiments 1-9 or 14-20, wherein R₆ is

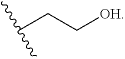 [SMILES: *CCO]

32. The compound of any of embodiments 1-9 or 14-20, wherein R₆ is

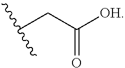 [SMILES: *CC(=O)O]

33. The compound of any of embodiments 1-9 or 14-20, wherein R₆ is

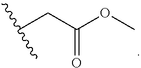 [SMILES: *CC(=O)OC]

34. The compound of any of embodiments 1-9 or 14-20, wherein R₆ is

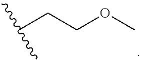 [SMILES: *CCOC]

35. A compound of Formula (II):

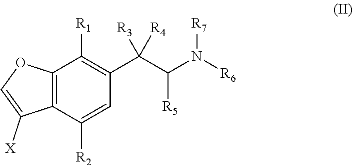

or a pharmaceutically acceptable salt thereof,
wherein,
X is halogen, deuterium, OH, OR₉, or CF₃;
   wherein R₉ is hydrogen, deuterium, halogen, alkyl, heteroalkyl, haloalkyl, O-alkyl, O-heteroalkyl, O-haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
   R₃ and R₄ together are carbonyl; or
   R₃ and R₄ together with the carbon atom to which they are attached form a cycloalkyl;
R₅ is hydrogen, deuterium, halogen, alkyl, heteroalkyl, haloalkyl, O-alkyl, O-heteroalkyl, or O-haloalkyl; and
R₆ and R₇ are independently hydrogen, deuterium, alkyl, or haloalkyl, cycloalkyl, or heterocyclyl; or
   one of R₆ or R₇ is

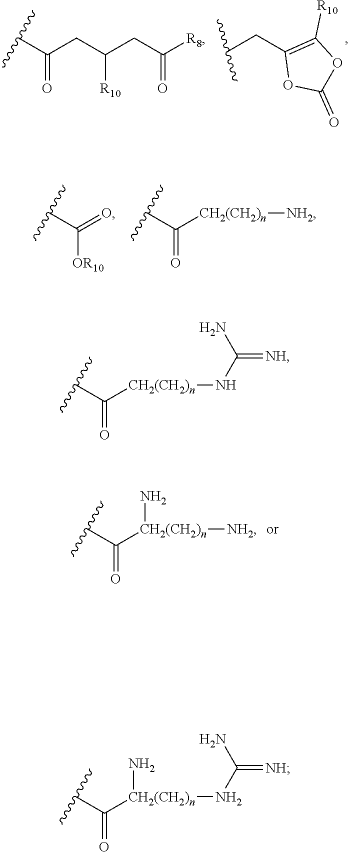

wherein R₁₀ and R₈ are alkyl; and
wherein n is an integer from 2 to 5.
36. The compound of embodiment 35, wherein
R₁ and R₂ are independently C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl;
R₃ and R₄ are independently C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together with the carbon atom to which they are attached form a C₃-C₇ cycloalkyl;
R₅ is C₁-C₆ alkyl, C₁-C₆ heteroalkyl, C₁-C₆ haloalkyl, O-alkyl, O-heteroalkyl, or O-haloalkyl; or
R₆ and R₇ are independently C₁-C₆ alkyl, C₁-C₆ haloalkyl, C₃-C₇ cycloalkyl, or C₃-C₇ heterocyclyl; or
   one of R₆ or R₇ is

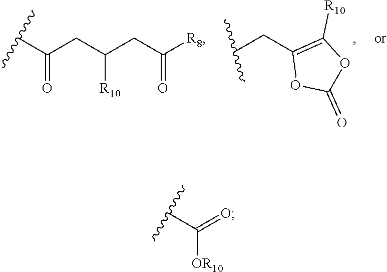

wherein R₁₀ and R₈ are independently C₁-C₆ alkyl.
37. The compound of any of embodiments 35-36, wherein X is F.
38. The compound of any of embodiments 35-37, wherein R₁ and R₂ are hydrogen.
39. The compound of any of embodiments 35-38, wherein R₅ is C₁-C₆ alkyl.
40. The compound of embodiment 39, wherein R₅ is methyl.
41. The compound of embodiment 39, wherein R₅ is ethyl.
42. The compound of any of embodiments 35-41, wherein R₆ is C₁-C₆ alkyl and R₇ is hydrogen.
43. The compound of embodiment 42, wherein R₆ is methyl.
44. The compound of embodiment 42, wherein R₆ is ethyl.
45. The compound of claim any of embodiments 35-41, wherein R₆ and R₇ are hydrogen.
46. The compound of any of embodiments 35-45, wherein R₃ and R₄ are hydrogen.
47. The compound of any of embodiments 35-45, wherein R₃ and R₄ together are carbonyl.
48. The compound of any of embodiments 35-45, wherein R₃ is OH.
49. The compound of any of embodiments 35-45, wherein R₃ and R₄ are deuterium.
50. The compound of any of embodiments 35-45, wherein R₃ is halogen.
51. The compound of embodiment 50, wherein R₃ is F.
52. The compound of embodiment 51, wherein R₄ is halogen.
53. The compound of embodiment 52, wherein R₄ is F.
54. The compound of any of embodiments 35-53, wherein R₆ is

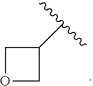 [SMILES: *C1COC1]

55. The compound of any of embodiments 35-53, wherein R₆ is

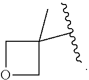 [SMILES: *C1(C)COC1]

56. The compound of any of embodiments 35-53, wherein R₆ is

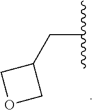 [SMILES: *CC1COC1]

57. The compound of any of embodiments 35-53, wherein R₆ is

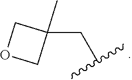 [SMILES: *CC1(C)COC1]

58. The compound of any of embodiments 35-53, wherein R₆ is

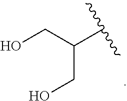 [SMILES: *C(CO)CO]

59. The compound of any of embodiments 35-53, wherein R₆ is

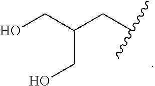 [SMILES: *CC(CO)CO]

60. The compound of any of embodiments 35-53, wherein R₆ is

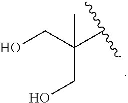 [SMILES: *C(C)(CO)CO]

61. The compound of any of embodiments 35-53, wherein R₆ is

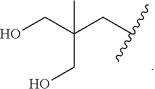 [SMILES: *CC(C)(CO)CO]

62. The compound of any of embodiments 35-53, wherein R₆ is

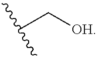 [SMILES: *CO]

63. The compound of any of embodiments 35-53, wherein R₆ is

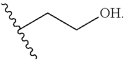 [SMILES: *CCO]

64. The compound of any of embodiments 35-53, wherein R₆ is

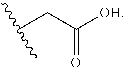 [SMILES: *CC(=O)O]

65. The compound of any of embodiments 35-53, wherein R₆ is

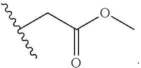 [SMILES: *CC(=O)OC]

66. The compound of any of embodiments 35-53, wherein R₆ is

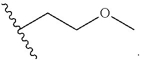 [SMILES: *CCOC]

67. A compound of Formula (I-B):

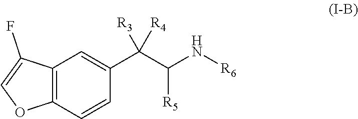

or a pharmaceutically acceptable salt thereof,
wherein,
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, C₁-C₆ haloalkyl, C₃-C₇ cycloalkyl, or C₃-C₇ heterocyclyl.
68. A compound of formula (II-B):

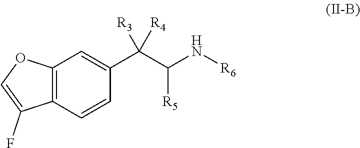

or a pharmaceutically acceptable salt thereof,
wherein,
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
   R₃ and R₄ together are carbonyl; or
   R₃ and R₄ together with the carbon atom to which they are attached form a C₃-C₇ cycloalkyl;
R₅ is hydrogen, deuterium, halogen, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, C₁-C₆ haloalkyl, C₃-C₇ cycloalkyl, or C₃-C₇ heterocyclyl.
69. A compound selected from:

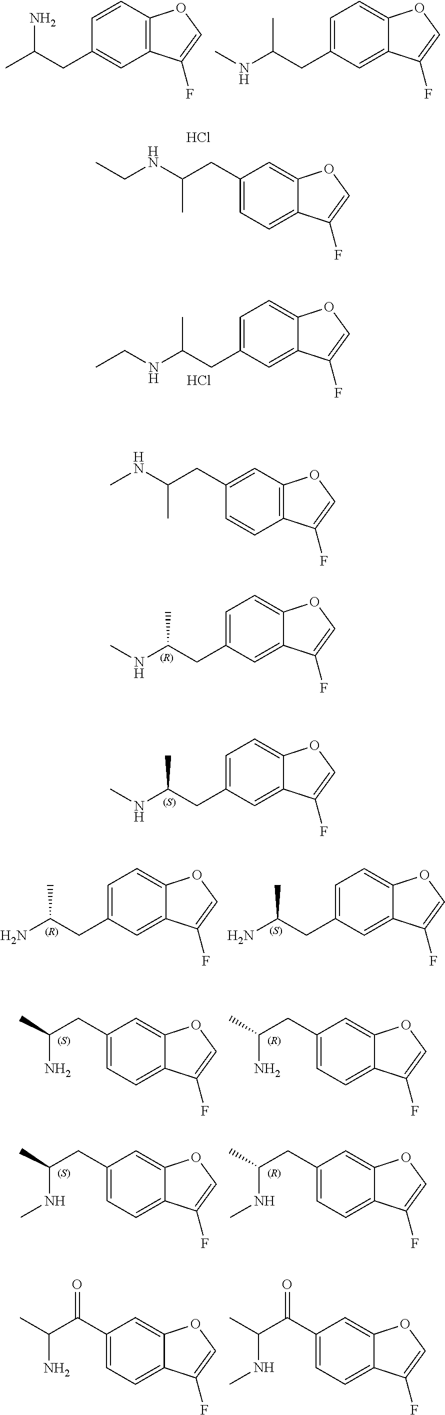

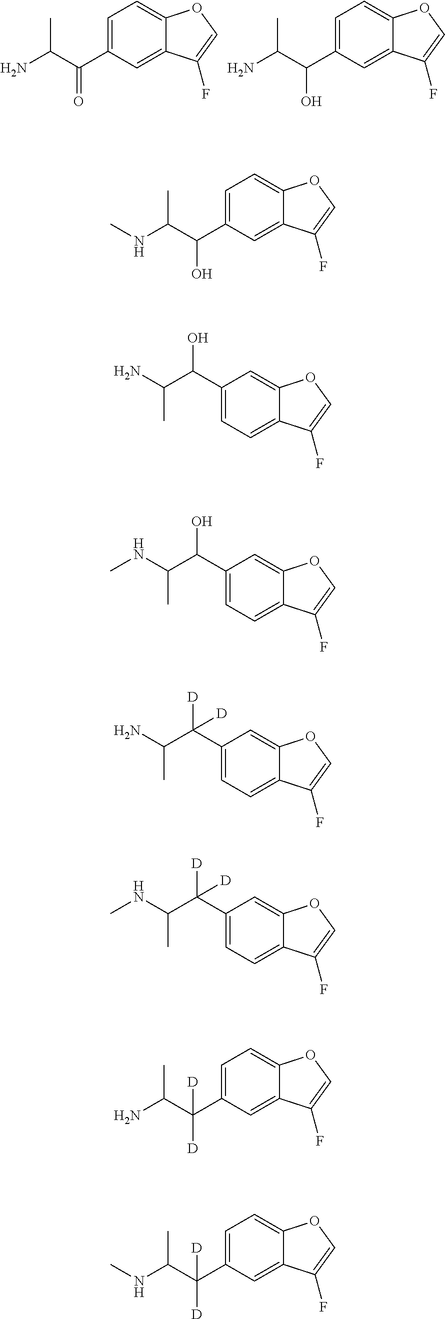

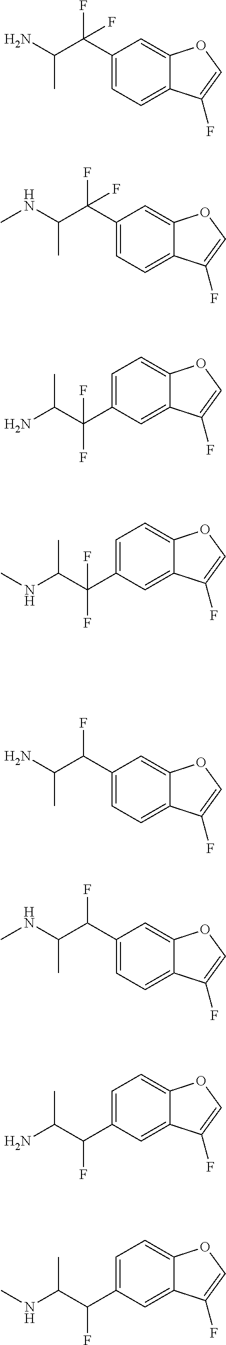

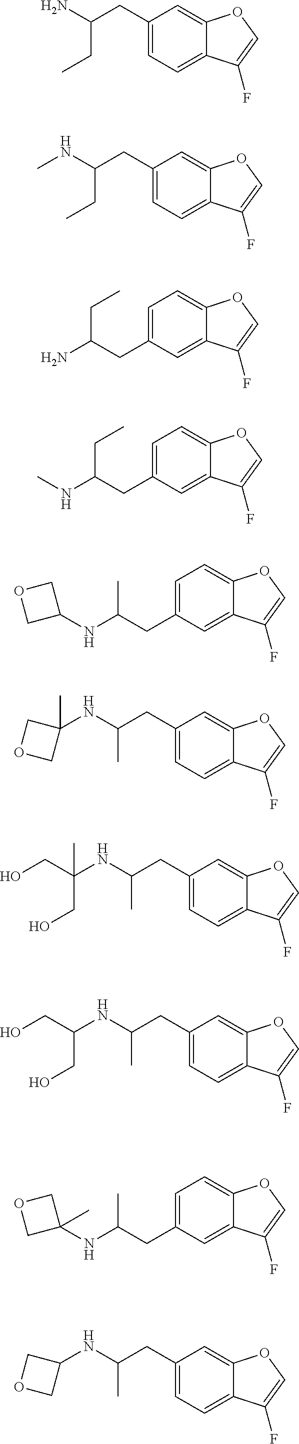

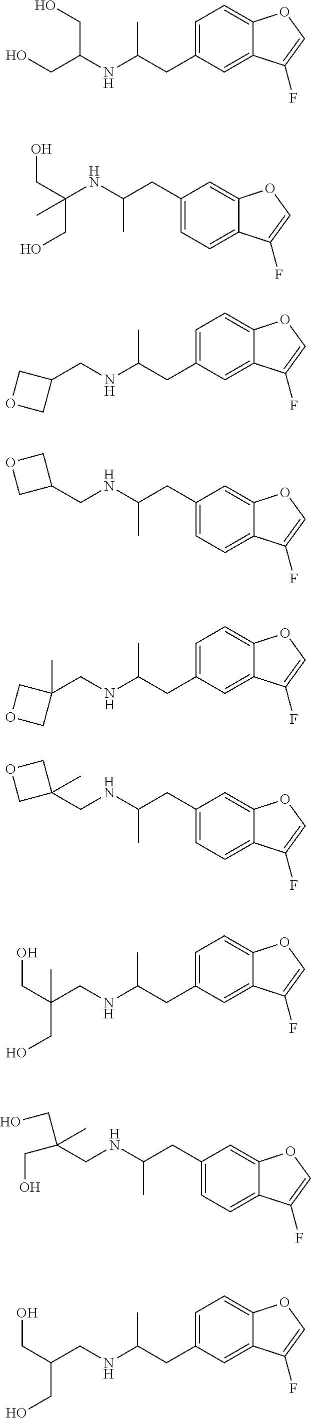

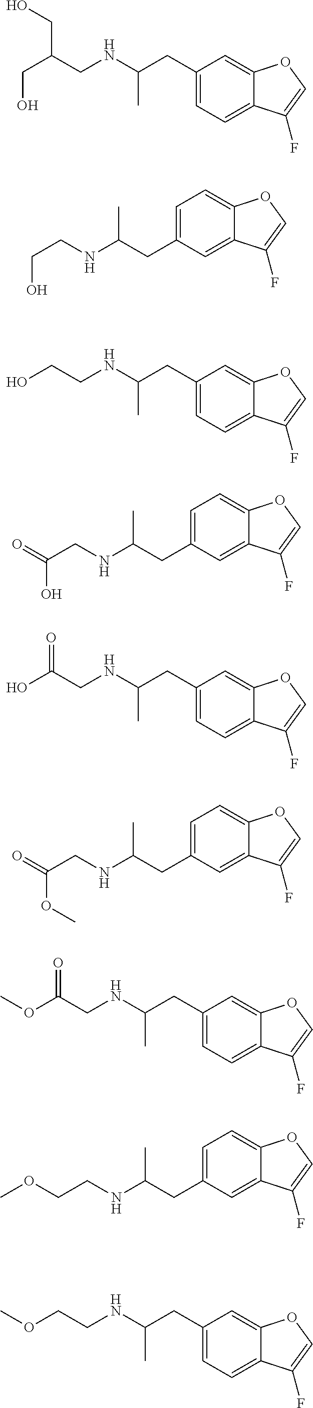

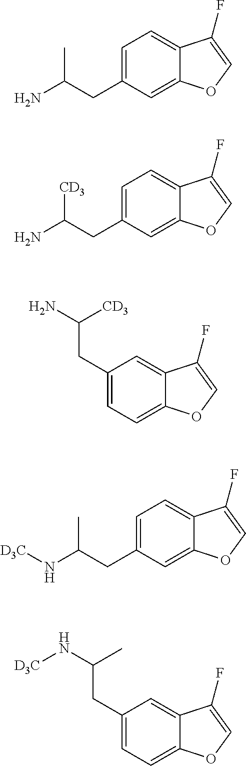 [SMILES: CC(N)Cc1ccc2c(F)coc2c1 ; [2H]C([2H])([2H])C(N)Cc1ccc2c(F)coc2c1 ; [2H]C([2H])([2H])C(N)Cc1ccc2occ(F)c2c1 ; [2H]C([2H])([2H])NC(C)Cc1ccc2c(F)coc2c1 ; [2H]C([2H])([2H])NC(C)Cc1ccc2occ(F)c2c1]

70. A composition comprising the compound of any of embodiments 1-69 and a pharmaceutically acceptable carrier.
71. A method of treating a neurological disorder, comprising administering to a subject in need thereof an effective amount of the compound of any of embodiments 1-69 or the composition of embodiment 70.
72. The method of embodiment 71, wherein the neurological disorder is post-traumatic stress disorder.
73. The method of embodiment 71, wherein the neurological disorder is an eating disorder including post-traumatic stress disorder.
74. The method of embodiment 71, wherein the neurological disorder is obsessive-compulsive disorder (OCD).

### Examples

The preparation of the compounds of the disclosure (e.g., a compound of Formula (I), (I-A), (I-B), (II), (II-A), or (II-B), or Table 1) is illustrated by the following examples, which are not to be construed as limiting the disclosure in scope or spirit to the specific procedures and compounds described in them.

### General Experimental

In general, the disclosed compounds are prepared by the method illustrated in the general reaction scheme described below, or by modifications thereof, using readily available starting materials, reagents, and conventional synthetic procedures. However, those skilled in the art will recognize that other methods may also be suitable. Also, in these reactions, it is possible to make use of variants that are in themselves known but are not mentioned here. Unless otherwise noted, all materials/reagents were obtained from commercial suppliers and used without further purification. Reactions were monitored by LC-MS and/or thin layer chromatography (TLC) on silica gel 60 F254 (0.2 mm) pre-coated aluminum foil or glass-backed and visualized using UV light. 1 HNMR (400 MHz) spectra was recorded on Bruker spectrometers at room temperature (RT) with TMS or residual solvent peak as internal standard. The line positions or multiples are given in (6) and coupling constants (J) are given as absolute values in Hertz (Hz). The multiplicities in 1 HNMR spectra are abbreviated as follows: s (singlet), d (doublet), t (triplet), q (quartet), m (multiplet), br or broad (broadened). Preparative HPLC purification was performed on Shimadzu LC-6AD. All purification work was complete using Shim-pack PREP-DDS (H) KIT column. The mobile phases were water (with 0.1% HCO₂H) and acetonitrile. All reagents used were HPLC grade. The flow rate was 10 mL/min.

Preparative TLC was performed on Whatman LK6F Silica Gel 60A size 20×20 cm plates with a thickness of 1000 μm or equivalent. LC-MS was performed on Shimadzu LCMS-2020 equipped with LC-20AD or 30AD pumps, SPD-M20A PDA and Alltech 3300 ELSD. Mobile Phase A: water (0.1% formic acid); Mobile Phase B: acetonitrile (ACN); Duration: 5 minutes; Column: Sepax BR-C18 4.6*50 mm, 3 μm; Flow Rate: 1.0 mL/min; Oven Temperature: 40° C.

### Embodiment A. Synthesis of Compounds 1 and 2: Representative Compounds 1 and 2 can be Made Via Following Steps, Scheme 1

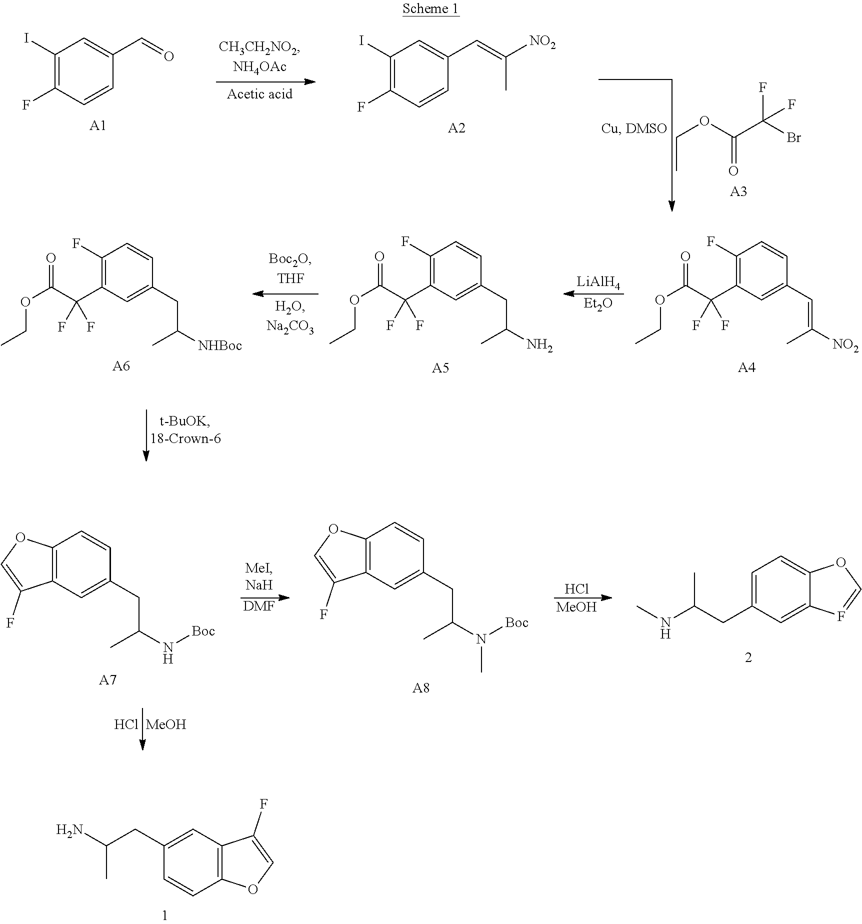

Synthesis of A2. To a stirred mixture of 4-fluoro-3-iodobenzaldehyde, A1 (4 g, 15.99 mmol, 1 equiv) in nitroethane (25 mL) were added ammonium acetate (1.23 g, 15.99 mmol, 1 equiv) and acetate (960.8 mg, 15.99 mmol, 1 equiv) dropwise/in portions at room temperature. The resulting mixture was stirred for additional overnight at 60° C. under argon atmosphere. The resulting mixture was concentrated under vacuum. The residue was purified by silica gel column chromatography, eluted with petroleum ether/ethyl acetate to afford 2.5 grams of A2 which was used directly for the next step without further characterization.

Synthesis of A4: The mixture of copper (1293.4 mg, 20.35 mmol, 2.5 equiv) and ethyl 2-bromo-2,2-difluoroacetate, A3 (4.1 g, 20.35 mmol, 2.5 equiv) in dimethyl sulfoxide (25 mL) was stirred for 30 minutes at room temperature under nitrogen atmosphere. To this reaction mixture, A2 (2.5 g, 8.14 mmol, 1 equiv) in dimethyl sulfoxide (5 ml) was added dropwise, then reaction was stirred at 50° C. for 15 hours. After completion of reaction, reaction was quenched with water (10 ml) and extracted with diethyl ether (50 ml). The organic layer was separated, filtered on celite, dried over sodium sulfate and concentrated under reduced pressure. The residue was purified by silica gel column chromatography, eluted with petroleum ether/ethyl acetate (15:1) to afford 1 g of A4 which was used directly for the next step without further characterization.

Synthesis of A5: A solution of A4 (500 mg, 1.65 mmol, 1 equiv) in diethyl ether (12.5 mL) was slowly added with continuous stirring to a solution of LiAlH₄ (250.30 mg, 6.60 mmol, 4 equiv) in diethyl ether (12.5 mL) under nitrogen atmosphere. After the addition, the solution was refluxed for 2 hours. The reaction was allowed to cool to 0° C. and quenched by slow addition of 0.1 mL of water, 0.1 mL of 15% sodium hydroxide aqueous solution and 0.3 ml of water. The lithium and aluminum salts were filtered off and washed with diethyl ether. The resulting solution was concentrated under vacuum. The residue was purified by reversed-phase flash chromatography with the following conditions: column, C₁₈ silica gel; mobile phase, acetonitrile in water (10 mmol/L NH₄HCO₃), 10% to 50% gradient in 15 min; detector, UV 210 nm. This resulted in 200 mg of A5; and this was used directly for the next step without further characterization.

Synthesis of A6: To a solution of A5 (200 mg, 0.86 mmol, 1 equiv) in tetrahydrofuran (3 mL) and water (3 mL) was added di-tert-butyl dicarbonate (205.9 mg, 0.94 mmol, 1.1 equiv) and sodium carbonate (227.21 mg, 2.145 mmol, 2.5 equiv). The mixture was stirred for one hour at room temperature. After completion of the reaction, the resulting mixture was poured into water and extracted with ethyl acetate. The combined organic layers were washed with water and brine, dried over sodium sulfate and concentrated under reduced pressure. The residue was purified by reversed-phase flash chromatography with the following conditions: column, C₁₈ silica gel; mobile phase, acetonitrile in water (0.05% NH₄HCO₃), 20% to 100% gradient in 15 min; detector, UV 210 nm. This resulted in 220 mg of A6, and this was used directly for the next step without further characterization.

Synthesis of A7: To a solution of A6 (190 mg, 0.57 mmol, 1 equiv) in tetrahydrofuran (1.9 mL) was added tert-butoxypotassium (95.9 mg, 0.85 mmol, 1.5 equiv) and 18-crown-6 (75.3 mg, 0.28 mmol, 0.5 equiv) under nitrogen atmosphere. The mixture was stirred for 3 hours at 80° C. Then, it was cooled to room temperature and was poured into water and extracted with ethyl acetate. The combined organic layers were washed with water and brine, dried over sodium sulfate and concentrated under reduced pressure. The residue was purified by reversed-phase flash chromatography with the following conditions: column, C₁₈ silica gel; mobile phase, acetonitrile in water (0.05% NH₄HCO₃), 20% to 100% gradient in 15 min; detector, UV 210 nm. This resulted in 70 mg of A7, which was used directly for the next step without further characterization.

Synthesis of A8: To a solution of A7 (110 mg, 0.375 mmol, 1 equiv) in dimethylformamide (2.2 mL) was added sodium hydride (22.5 mg, 0.56 mmol, 1.5 equiv, 60% in oil) at 0° C. The mixture was stirred for 15 minutes. Methyl iodide (106.5 mg, 0.75 mmol, 2 equiv) was added and the mixture was allowed to warm to 50° C. and stirred for 1 hour. The reaction mixture was quenched by saturated NH₄Cl aqueous and extracted with ethyl acetate (2×10 mL). The combined organic layers were washed with water and brine, dried over sodium sulfate and concentrated under reduced pressure. The residue was purified by reversed-phase flash chromatography with the following conditions: column, C₁₈ silica gel; mobile phase, acetonitrile in water (0.05% NH₄HCO₃), 20% to 100% gradient in 15 min; detector, UV 210 nm. This resulted in 56 mg of A8, which was used directly for the next step without further characterization.

Synthesis of Compound 2,1-(3-fluorobenzofuran-5-yl)-N-methylpropan-2-amine: To HCl in methanol (2 mol/L, 1.1 mL) was added A8 (56 mg, 0.18 mmol, 1 equiv). The solution was stirred for 3 hours at room temperature. The solvent was removed under reduced pressure and the product was triturated by the addition of diethyl ether. This resulted in 25.5 mg of 2, 1-(3-fluorobenzofuran-5-yl)-N-methylpropan-2-amine (25.5 mg). MS m/z [M+H]⁺ (ESI): 208.05. ¹H NMR (300 MHz, Methanol-d₄) δ 7.87 (d, J=4.5 Hz, 1H), 7.56 (d, J=1.5 Hz, 1H), 7.51-7.45 (m, 1H), 7.35-7.27 (m, 1H), 3.62-3.45 (m, 1H), 3.30-3.23 (m, 1H), 2.96-2.86 (m, 1H), 2.75 (s, 3H), 1.26 (d, J=6.3 Hz, 3H).

Synthesis of Compound 1,1-(3-fluorobenzofuran-5-yl)propan-2-amine: To HCl in methanol (2 mol/L, 0.4 mL) was added A7 (20 mg, 0.07 mmol, 1 equiv). The solution was stirred for 3 hours at room temperature. The solvent was removed under reduced pressure. The residue was purified by reversed-phase flash chromatography with the following conditions: column, C₁₈ silica gel; mobile phase, acetonitrile in water (0.1% HCl), 5% to 50% gradient in 15 min; detector, UV 210 nm. This resulted in 6.8 mg of 1, 1-(3-fluorobenzofuran-5-yl)propan-2-amine (6.8 mg). MS m/z [M+H]⁺ (ESI): 194.10. ¹H NMR (300 MHz, Methanol-d₄) δ 7.87 (d, J=4.5 Hz, 1H), 7.54 (s, 1H), 7.51-7.43 (m, 1H), 7.33-7.26 (m, 1H), 3.66-3.49 (m, 1H), 3.15-3.05 (m, 1H), 3.00-2.91 (m, 1H), 1.29 (d, J=6.6 Hz, 3H).

### Embodiment B. Synthesis of Compounds 3 and 5: Representative Compounds 3 and 5 can be Made Via Following Steps, Scheme 2

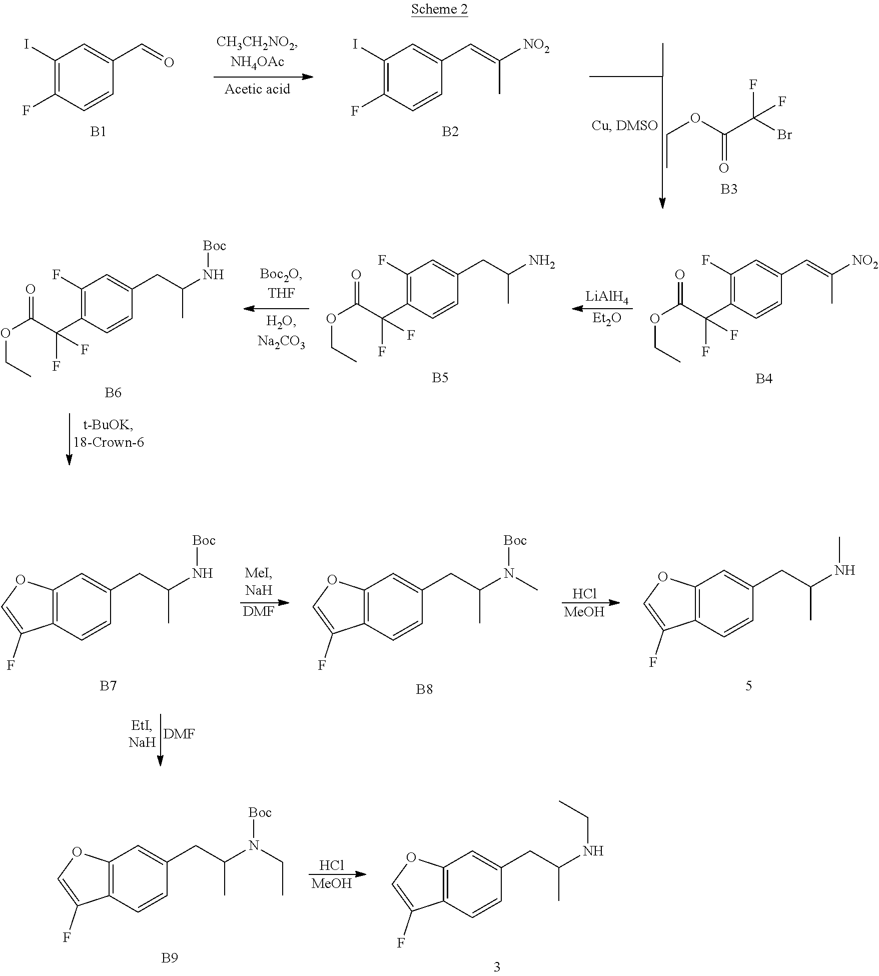

Synthesis of B2: A solution of 3-fluoro-4-iodobenzaldehyde (10 g, 39.99 mmol, 1 equiv) in nitroethane (100 mL) was treated with NH₄OAc (3.1 g, 39.99 mmol, 1.00 equiv) under nitrogen atmosphere followed by the addition of acetic acid (2.4 g, 39.99 mmol, 1.00 equiv) dropwise at room temperature. The resulting mixture was stirred for overnight at 60° C. under argon atmosphere. The mixture was concentrated under reduced pressure. The residue was purified by silica gel column chromatography, eluted with dichloromethane/petroleum ether (1:1) to afford B2 (9 g). This crude product was directly for the next step without further purification and characterization.

Synthesis of B4: The mixture of copper (1.8 g, 28.50 mmol, 2.5 equiv) and ethyl 2-bromo-2,2-difluoroacetate (5.8 g, 28.50 mmol, 2.5 equiv) in dimethyl sulfoxide (35 mL) was stirred for 30 minutes at room temperature under nitrogen atmosphere. To this reaction mixture, B2 (3.5 g, 11.40 mmol, 1 equiv) in dimethyl sulfoxide (5 ml) was added dropwise, then reaction was stirred at 50° C. for 15 hours. After completion of reaction, reaction was quenched with water (10 ml) and extracted with diethyl ether (50 ml). The organic layer was separated, filtered on celite, dried over sodium sulfate and concentrated under reduced pressure. The residue was purified by silica gel column chromatography, eluted with petroleum ether/ethyl acetate (15:1) to afford B4 (1.4 g, 41%).

Synthesis of B5: A solution of B4 (1.4 g, 6.00 mmol, 1 equiv) in diethyl ether (35 mL) was slowly added with continuous stirring to a solution of LiAlH₄ (911.2 mg, 24.01 mmol, 4 equiv) in diethyl ether (35 mL) under nitrogen atmosphere. After the addition, the solution was refluxed for 2 hours. The reaction was allowed to cool to 0° C. and quenched by slow addition of 0.2 mL of water, 0.2 mL of 15% sodium hydroxide aqueous solution and 0.6 ml of water. The lithium and aluminum salts were filtered off and washed with diethyl ether. The resulting solution was concentrated under vacuum. The residue was purified by reversed-phase flash chromatography with the following conditions: column, C₁₈ silica gel; mobile phase, acetonitrile in water (0.05% NH₄HCO₃), 10% to 50% gradient in 15 min; detector, UV 210 nm. This resulted in B5 (700 mg), which was used for the next step without further purification.

Synthesis of B6: To a solution of B5 (700 mg, 3.00 mmol, 1 equiv) in tetrahydrofuran (10.5 mL) and water (10.5 mL) was added di-tert-butyl dicarbonate (720.53 mg, 3.301 mmol, 1.1 equiv) and sodium carbonate (795.3 mg, 7.50 mmol, 2.5 equiv). The mixture was stirred for one hour at room temperature. After completion of the reaction, the resulting mixture was poured into water and extracted with ethyl acetate. The combined organic layers were washed with water and brine, dried over sodium sulfate and concentrated under reduced pressure. The residue was purified by reversed-phase flash chromatography with the following conditions: column, C₁₈ silica gel; mobile phase, acetonitrile in water (0.05% NH₄HCO₃), 20% to 100% gradient in 15 min; detector, UV 210 nm. This resulted 800 mg of B6, which was used for the next step without further purification.

Synthesis of B7: To a solution of B6 (550 mg, 1.65 mmol, 1 equiv) in tetrahydrofuran (5.5 mL) was added tert-butoxypotassium (277.7 mg, 2.48 mmol, 1.5 equiv) and 18-crown-6 (218.1 mg, 0.83 mmol, 0.5 equiv) under nitrogen atmosphere. The mixture was stirred for 3 hours at 80° C. Then, it was cooled to room temperature and was poured into water and extracted with ethyl acetate. The combined organic layers were washed with water and brine, dried over sodium sulfate and concentrated under reduced pressure. The residue was purified by reversed-phase flash chromatography with the following conditions: column, C₁₈ silica gel; mobile phase, acetonitrile in water (0.05% NH₄HCO₃), 20% to 100% gradient in 15 min; detector, UV 210 nm. This resulted 50 mg of B7, which was used for the next step without further purification.

Synthesis of B8: To a solution of B7 (50 mg, 0.170 mmol, 1 equiv) in dimethylformamide (1 mL) was added sodium hydride (10.2 mg, 0.26 mmol, 1.5 equiv, 60%) at 0° C. The mixture was stirred for 15 min. methyl iodide (72.6 mg, 0.51 mmol, 3 equiv) was added and the mixture was allowed to warm to 50° C. and stirred for 2 h. The reaction mixture was quenched by saturated NH₄Cl aqueous and extracted with ethyl acetate (2×10 mL). The combined organic layers were washed with water and brine, dried over sodium sulfate and concentrated under reduced pressure. The residue was purified by reversed-phase flash chromatography with the following conditions: column, C₁₈ silica gel; mobile phase, acetonitrile in water (0.05% NH₄HCO₃), 20% to 100% gradient in 15 min; detector, UV 210 nm. This resulted in 30 mg of B8, which was used for the next step without further purification.

Synthesis of Compound 5, 1-(3-fluorobenzofuran-6-yl)-N-methylpropan-2-amine: To HCl in methanol (4 mol/L, 0.3 mL) was added B8 (30 mg, 0.01 mmol, 1 equiv). The solution was stirred for 1 hours at room temperature. The solvent was removed under reduced pressure and the product was triturated by the addition of diethyl ether. This resulted 18.6 mg of the desired product 5, 1-(3-fluorobenzofuran-6-yl)-N-methylpropan-2-amine. MS m/z [M+H]⁺ (ESI): 208.00. ¹H NMR (300 MHz, Methanol-d₄) δ 7.85 (d, J=4.5 Hz, 1H), 7.62 (d, J=8.0 Hz, 1H), 7.49-7.43 (m, 1H), 7.31-7.23 (m, 1H), 3.59-3.46 (m, 1H), 3.30-3.24 (m, 1H), 2.96-2.85 (m, 1H), 2.74 (s, 3H), 1.26 (d, J=6.6 Hz, 3H).

Synthesis of B9: To a solution of B7 (40 mg, 0.14 mmol, 1 equiv) in dimethylformamide (0.8 mL) was added sodium hydride (8.2 mg, 0.20 mmol, 1.5 equiv, 60%) at 0° C. The mixture was stirred for 30 minutes at 30° C. Ethyl iodide (63.8 mg, 0.41 mmol, 3 equiv) was added and the mixture was allowed to warm to 50° C. and stirred for 2 hours. The reaction mixture was quenched by saturated NH₄Cl aqueous and extracted with ethyl acetate (2×10 mL). The combined organic layers were washed with water and brine, dried over sodium sulfate and concentrated under reduced pressure. The residue was purified by reversed-phase flash chromatography with the following conditions: column, C₁₈ silica gel; mobile phase, acetonitrile in water (0.05% NH₄HCO₃), 20% to 100% gradient in 15 min; detector, UV 210 nm. This resulted 30 mg of B9, which was used for the next step without further purification.

Synthesis of Compound 3, N-ethyl-1-(3-fluorobenzofuran-6-yl)propan-2-amine hydrochloride: To HCl in methanol (4 mol/L, 0.6 mL) was added B9 (30 mg, 0.09 mmol, 1 equiv). The solution was stirred for 1 hours at room temperature. The solvent was removed under reduced pressure and the product was triturated by the addition of diethyl ether. This resulted 10.3 mg of 3, N-ethyl-1-(3-fluorobenzofuran-6-yl)propan-2-amine hydrochloride. MS m/z [M+H]⁺ (ESI): 222.10. ¹H NMR (300 MHz, Methanol-d₄) δ 7.86 (d, J=4.5 Hz, 1H), 7.62 (d, J=8.1 Hz, 1H), 7.44 (s, 1H), 7.28-7.20 (m, 1H), 3.64-3.47 (m, 1H), 3.35-3.27 (m, 1H), 3.22-3.04 (m, 2H), 2.89-2.77 (m, 1H), 1.34 (t, J=7.3 Hz, 3H), 1.23 (d, J=6.6 Hz, 3H).

### Biological Assays

Serotonin transporter (SERT) Uptake Inhibition: Compound activity was assessed using an in vitro assay. Briefly, HEK cells expressing recombinant SERT were plated at 50,000 cells/well on a 96 well plate pre-coated with Matrigel one day prior to the experiment. Culture medium (DMEM/FBS) was removed from each well, and 30 μl of assay buffer (Tris-HCl 50 mM, EDTA 4 mM, BDA 0.1%) with the desired concentration of test compound was added. The plate was incubated at 37° C. for 15 minutes. Assay buffer (30 μl) containing the same concentration of compound diluted in [3H]-Serotonin uptake buffer (final concentration 10 nM) was added to each well and the plate was incubated at 37° C. for 5 minutes. The reaction mixture was removed, and the cells were washed (2×) with 100 μl ice-cold assay buffer. Lysis buffer (50 μl) was added to the cells followed by a 5 minute incubation with gentle shaking at room temperature. The lysate was transferred to a 96 well isoplate. Optiphase supermix (100 μl) was added to each well and was thoroughly mixed. Radioactivity was counted with Microbeta Counter and reported as counts per minute (CPM). Fluoxetine was used as a control to measure non-specific uptake and for data normalization. Percent inhibition of [3H]-Serotonin uptake is based on the calculation: 100×(1−(CPMtest sample−CPMnon-specific uptake)/(CPMMAX−CPMMIN)).

Dopamine transporter (DAT) Uptake Inhibition: Compound activity was assessed using a convenient in vitro assay. Briefly, HEK cells expressing recombinant DAT were plated at 50,000 cells/well on a 96 well plate pre-coated with Matrigel one day prior to the experiment. Culture medium (DMEM/FBS) was removed and 30 μl of assay buffer (Tris-HCl 50 mM, EDTA 4 mM, BDA 0.1%) with the desired concentration of test compound was added. The plate was incubated at 37° C. for 15 minutes. Assay buffer (30 μl) containing the same concentration of compound diluted in [3H]-Dihydroxyphenylethylamine (dopamine) uptake buffer (final concentration 20 nM) was added to each well and the plate was incubated at 37° C. for 5 minutes. The reaction mixture was removed, and the cells were washed with 100 μl ice-cold assay buffer twice. Lysis buffer (50 μl) was added to the cells followed by a 5 minute incubation with gentle shaking at room temperature. The lysate was transferred to a 96 well isoplate. Optiphase supermix (100 μl) was added to each well with complete mixing. Radioactivity was counted with Microbeta Counter and reported as CPM. Nomifensine was used as a control to measure non-specific uptake and for data normalization. Percent inhibition of [3H]-Dopamine uptake calculations: 100×(1−(CPMtest sample−CPMnon-specific uptake)/(CPMMAX−CPMMIN)).

Norepinephrine transporter (NET) Uptake Inhibition: Compound activity was assessed using a convenient in vitro assay. Briefly, HEK cells expressing recombinant NET were plated at 50,000 cells/well on a 96 well plate pre-coated with Matrigel one day prior to the experiment. Culture medium (DMEM/FBS) was removed and 30 μl of assay buffer (Tris-HCl 50 mM, EDTA 4 mM, BDA 0.1%) with the desired concentration of test compound was added. The plate was incubated at 37° C. for 15 minutes. Assay buffer (30 μl) containing the same concentration of compound diluted in [3H]-Norepinephrine uptake buffer (final concentration 20 nM) was added to each well and the plate was incubated at 37° C. for 5 minutes. The reaction mixture was removed, and the cells were washed with 100 μl ice-cold assay buffer twice. Lysis buffer (50 μl) was added to the cells followed by a 5 minute incubation with gentle shaking at room temperature. The lysate was transferred to a 96 well isoplate. Optiphase supermix (100 μl) was added to each well with complete mixing. Radioactivity was counted with Microbeta Counter and reported as CPM. Desipramine was used as a control to measure non-specific uptake and for data normalization. Percent inhibition of [3H]-Norepinephrine uptake calculations: 100×(1−(CPMtest sample−CPMnon-specific uptake)/(CPMMAX−CPMMIN)).

TABLE 2
Modulators of Monoamine Transporter Uptake.

| | | SERT | SERT | SERT | DAT | NET |
|---|---|---|---|---|---|---|
| | | UPTAKE | UPTAKE | UPTAKE | UPTAKE | UPTAKE |
| | | INHIBITION | INHIBITION | INHIBITION | INHIBITION | INHIBITION |
| Compound | Structure | (%) @ 100 μM | (%) @ 10 μM | IC50 (μM) | IC50 (μM) | IC50 (μM) |
| 1 | 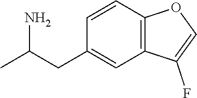 | 97.94 | 72.68 | 6.75 | 15.63 | 4.73 |
| 2 | 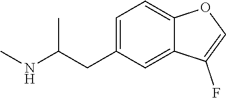 | 99.01 | 64.74 | 5.72 | 9.59 | 5.02 |
| MDMA | 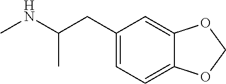 | 98.01 | 53.32 | 3.35 | 5.25 | 1.42 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of Formula (I): 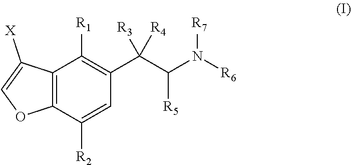
or a pharmaceutically acceptable salt thereof,
wherein,
X is halogen, deuterium, OH, OR₉, or CF₃;
wherein R₉ is hydrogen, deuterium, halogen, alkyl, heteroalkyl, haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
R₃ and R₄ together are carbonyl; or
R₃ and R₄ together with the carbon atom to which they are attached form a cycloalkyl;
R₅ is hydrogen, deuterium, halogen, alkyl, deuterated alkyl, heteroalkyl, haloalkyl, O-alkyl, O-heteroalkyl, or O-haloalkyl; and
R₆ and R₇ are independently hydrogen, deuterium, alkyl, allyl, deuterated alkyl, haloalkyl, cycloalkyl, or heterocyclyl, wherein the alkyl, deuterated alkyl, haloalkyl, cycloalkyl, or heterocyclyl are independently optionally substituted; or
one of R₆ or R₇ is 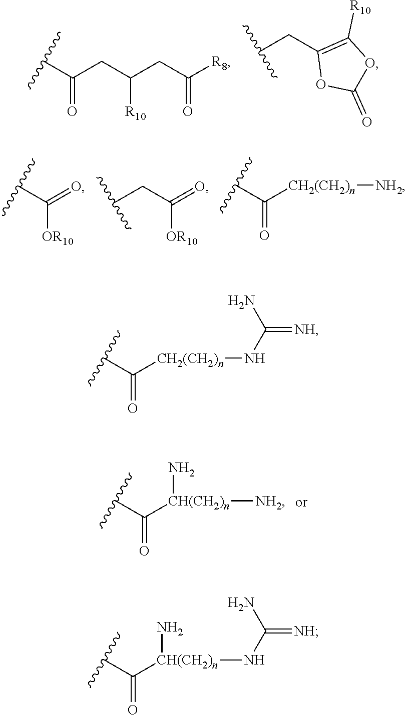
wherein R₁₀ and R₈ are alkyl or hydrogen; and
wherein n is an integer from 2 to 5.

2. The compound of claim 1, wherein
R₁ and R₂ are independently C₁-C₆ alkyl, C₁-C₆ heteroalkyl, C₁-C₆ haloalkyl, O—C₁-C₆ alkyl, 0-C₁-C₆ heteroalkyl, or O—C₁-C₆ haloalkyl;
R₃ and R₄ are independently C₁-C₆ alkyl, C₁-C₆ heteroalkyl, C₁-C₆ haloalkyl, O—C₁-C₆ alkyl, 0-C₁-C₆ heteroalkyl, or O—C₁-C₆ haloalkyl; or
R₃ and R₄ together with the carbon atom to which they are attached form a C₃-C₇ cycloalkyl;
R₅ is C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ and R₇ are independently C₁-C₆ alkyl, C₁-C₆ allyl, C₁-C₆ haloalkyl, C₃-C₇ cycloalkyl, C₃-C₇ heterocyclyl, wherein the alkyl, deuterated alkyl, haloalkyl, cycloalkyl, or heterocyclyl are independently optionally substituted; or
one of R₆ or R₇ is 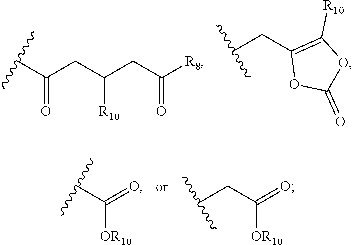
wherein R₁₀ and R₈ are independently C₁-C₆ alkyl.

3. The compound of claim 1, wherein X is F.

4. The compound of claim 1, wherein R₁ and R₂ are hydrogen.

5. The compound of claim 1, wherein R₃ and R₄ are hydrogen.

6. The compound of claim 1, wherein R₅ is C₁-C₆ alkyl.

7. The compound of claim 6, wherein R₅ is methyl.

8. The compound of claim 6, wherein R₅ is ethyl.

9. The compound of claim 6, wherein R₅ is hydrogen.

10. The compound of claim 1, wherein R₆ is C₁-C₆ alkyl and R₇ is hydrogen.

11. The compound of claim 10, wherein R₆ is methyl.

12. The compound of claim 10, wherein R₆ is ethyl.

13. The compound of claim 1, wherein R₆ and R₇ are hydrogen.

14. The compound of claim 1, wherein R₃ and R₄ together are carbonyl.

15. The compound of claim 1, wherein R₃ is OH.

16. The compound of claim 1, wherein R₃ and R₄ are deuterium.

17. The compound of claim 1, wherein R₃ is halogen.

18. The compound of claim 17, wherein R₃ is F.

19. The compound of claim 17, wherein R₄ is halogen.

20. The compound of claim 19, wherein R₄ is F.

21. The compound of claim 1, wherein R₆ is heterocyclyl.

22. The compound of claim 21, wherein R₆ is

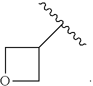 [SMILES: *C1COC1]

23. The compound of claim 21, wherein R₆ is

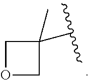 [SMILES: *C1(C)COC1]

24. The compound of claim 21, wherein R₆ is

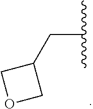 [SMILES: *CC1COC1]

25. The compound of claim 21, wherein R₆ is

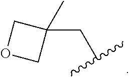 [SMILES: *CC1(C)COC1]

26. The compound of claim 1, wherein R₆ is

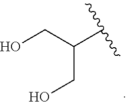 [SMILES: *C(CO)CO]

27. The compound of claim 1, wherein R₆ is

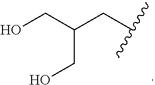 [SMILES: *CC(CO)CO]

28. The compound of claim 1, wherein R₆ is

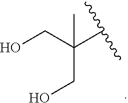 [SMILES: *C(C)(CO)CO]

29. The compound of claim 1, wherein R₆ is

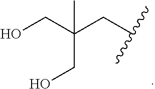 [SMILES: *CC(C)(CO)CO]

30. The compound of claim 1, wherein R₆ is

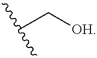 [SMILES: *CO]

31. The compound of claim 1, wherein R₆ is

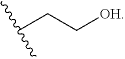 [SMILES: *CCO]

32. The compound of claim 1, wherein R₆ is

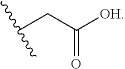 [SMILES: *CC(=O)O]

33. The compound of claim 1, wherein R₆ is

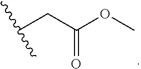 [SMILES: *CC(=O)OC]

34. The compound of claim 1, wherein R₆ is

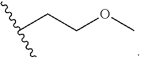 [SMILES: *CCOC]

35. The compound of claim 1, wherein the compound is

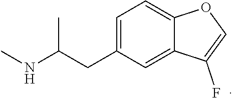

36. The compound of claim 35, wherein the compound is

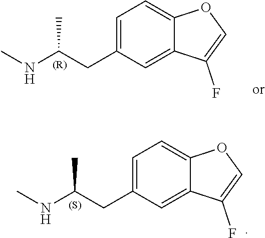

37. A compound of Formula (II): 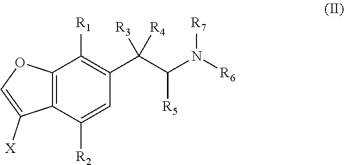
or a pharmaceutically acceptable salt thereof,
wherein,
X is halogen, deuterium, OH, OR₉, or CF₃;
wherein R₉ is hydrogen, deuterium, halogen, alkyl, heteroalkyl, haloalkyl, aryl, or benzyl;
R₁ and R₂ are independently hydrogen, deuterium, halogen, alkyl, heteroalkyl, haloalkyl, O-alkyl, O-heteroalkyl, O-haloalkyl, aryl, or benzyl;
R₃ and R₄ are independently hydrogen, deuterium, halogen, OH, alkyl, heteroalkyl, haloalkyl, O-alkyl, O-heteroalkyl, or O-haloalkyl; or
R₃ and R₄ together are carbonyl; or
R₃ and R₄ together with the carbon atom to which they are attached form a cycloalkyl;
R₅ is hydrogen, deuterium, halogen, alkyl, deuterated alkyl, heteroalkyl, haloalkyl, O-alkyl, O-heteroalkyl, or O-haloalkyl; and
R₆ and R₇ are independently hydrogen, deuterium, alkyl, allyl, haloalkyl, cycloalkyl, or heterocyclyl, wherein the alkyl, deuterated alkyl, haloalkyl, cycloalkyl, or heterocyclyl are independently optionally substituted; or
one of R₆ or R₇ is 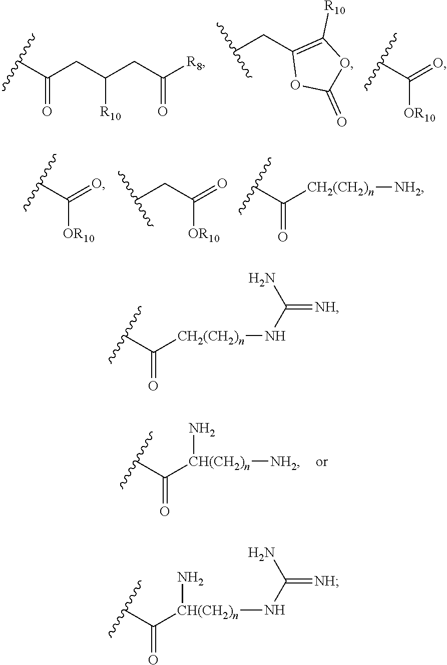
wherein R₁₀ and R₈ are alkyl or hydrogen; and
wherein n is an integer from 2 to 5.

38. The compound of claim 37, wherein
R₁ and R₂ are independently C₁-C₆ alkyl, C₁-C₆ heteroalkyl, C₁-C₆ haloalkyl, O—C₁-C₆ alkyl, 0-C₁-C₆ heteroalkyl, or O—C₁-C₆ haloalkyl;
R₃ and R₄ are independently C₁-C₆ alkyl, C₁-C₆ heteroalkyl, C₁-C₆ haloalkyl, O—C₁-C₆ alkyl, 0-C₁-C₆ heteroalkyl, or O—C₁-C₆ haloalkyl; or
R₃ and R₄ together with the carbon atom to which they are attached form a C₃-C₇ cycloalkyl;
R₅ is C₁-C₆ alkyl, C₁-C₆ heteroalkyl, C₁-C₆ haloalkyl, O-alkyl, O-heteroalkyl, or O-haloalkyl; or
R₆ and R₇ are independently C₁-C₆ alkyl, C₁-C₆ allyl, C₁-C₆ haloalkyl, C₃-C₇ cycloalkyl, or C₃-C₇ heterocyclyl, wherein the alkyl, deuterated alkyl, haloalkyl, cycloalkyl, or heterocyclyl are independently optionally substituted; or
one of R₆ or R₇ is 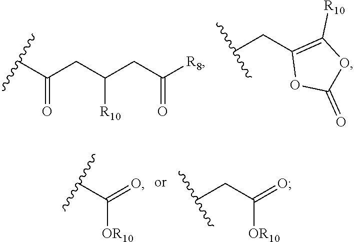
wherein R₁₀ and R₈ are independently C₁-C₆ alkyl or hydrogen.

39. The compound of claim 37, wherein X is F.

40. The compound of claim 37, wherein R₁ and R₂ are hydrogen.

41. The compound of claim 37, wherein R₅ is C₁-C₆alkyl.

42. The compound of claim 41, wherein R₅ is methyl.

43. The compound of claim 41, wherein R₅ is ethyl.

44. The compound of claim 37, wherein R₆ is C₁-C₆alkyl and R₇ is hydrogen.

45. The compound of claim 44, wherein R₆ is methyl.

46. The compound of claim 44, wherein R₆ is ethyl.

47. The compound of claim 37, wherein R₆ and R₇ are hydrogen.

48. The compound of claim 37, wherein R₃ and R₄ are hydrogen.

49. The compound of claim 37, wherein R₃ and R₄ together are carbonyl.

50. The compound of claim 37, wherein R₃ is OH.

51. The compound of claim 37, wherein R₃ and R₄ are deuterium.

52. The compound of claim 37, wherein R₃ is halogen.

53. The compound of claim 52, wherein R₃ is F.

54. The compound of claim 53, wherein R₄ is halogen.

55. The compound of claim 54, wherein R₄ is F.

56. The compound of claim 37, wherein R₆ is

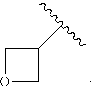 [SMILES: *C1COC1]

57. The compound of claim 37, wherein R₆ is

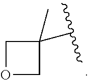 [SMILES: *C1(C)COC1]

58. The compound of claim 37, wherein R₆ is

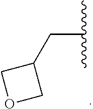 [SMILES: *CC1COC1]

59. The compound of claim 37, wherein R₆ is

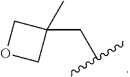 [SMILES: *CC1(C)COC1]

60. The compound of claim 37, wherein R₆ is

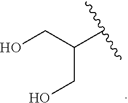 [SMILES: *C(CO)CO]

61. The compound of claim 37, wherein R₆ is

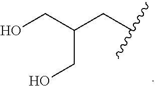 [SMILES: *CC(CO)CO]

62. The compound of claim 37, wherein R₆ is

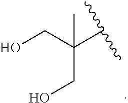 [SMILES: *C(C)(CO)CO]

63. The compound of claim 37, wherein R₆ is

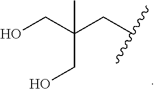 [SMILES: *CC(C)(CO)CO]

64. The compound of claim 37, wherein R₆ is

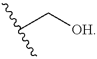 [SMILES: *CO]

65. The compound of claim 37, wherein R₆ is

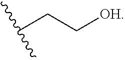 [SMILES: *CCO]

66. The compound of claim 37, wherein R₆ is

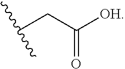 [SMILES: *CC(=O)O]

67. The compound of claim 37, wherein R₆ is

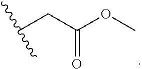 [SMILES: *CC(=O)OC]

68. The compound of claim 37, wherein R₆ is

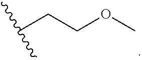 [SMILES: *CCOC]

69. The compound of claim 37, wherein the compound is

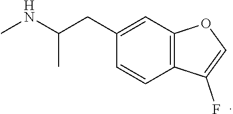

70. The compound of claim 69, wherein the compound is

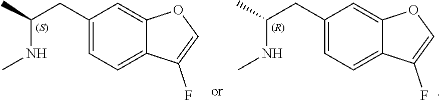

71. A compound of Formula (I-B): 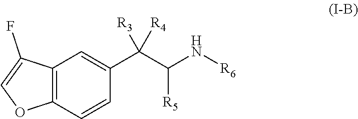
or a pharmaceutically acceptable salt thereof,
wherein,
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
R₃ and R₄ together are carbonyl; or
R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

72. A compound of formula (II-B): 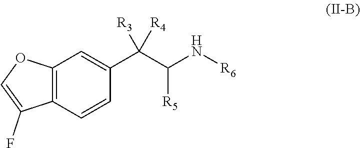
or a pharmaceutically acceptable salt thereof,
wherein,
R₃ and R₄ are independently hydrogen, deuterium, halogen, OH, C₁-C₆ alkyl, C₁-C₆ heteroalkyl, C₁-C₆ haloalkyl, O—C₁-C₆ alkyl, O—C₁-C₆ heteroalkyl, or O—C₁-C₆ haloalkyl; or
R₃ and R₄ together are carbonyl; or
R₃ and R₄ together with the carbon atom to which they are attached form a C₃-C₇ cycloalkyl;
R₅ is hydrogen, deuterium, halogen, C₁-C₆ alkyl, deuterated C₁-C₆ alkyl, C₁-C₆ heteroalkyl, C₁-C₆haloalkyl, O—C₁-C₆ alkyl, O—C₁-C₆ heteroalkyl, or O—C₁-C₆ haloalkyl; and
R₆ is hydrogen, deuterium, C₁-C₆ alkyl, deuterated C₁-C₆ alkyl, C₁-C₆ haloalkyl, C₃-C₇ cycloalkyl, or C₃-C₇ heterocyclyl.

73. A compound selected from:

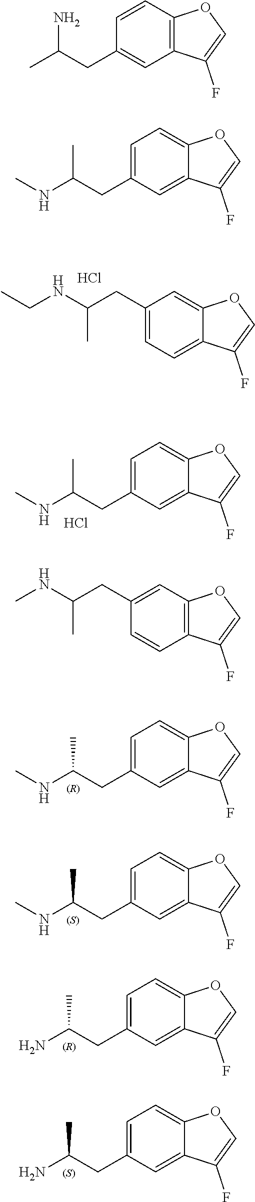

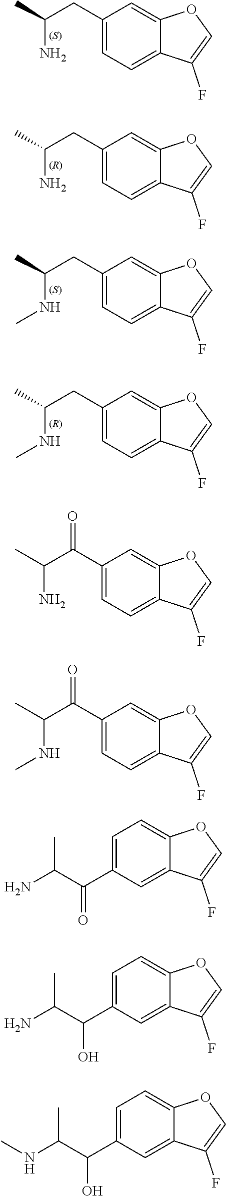

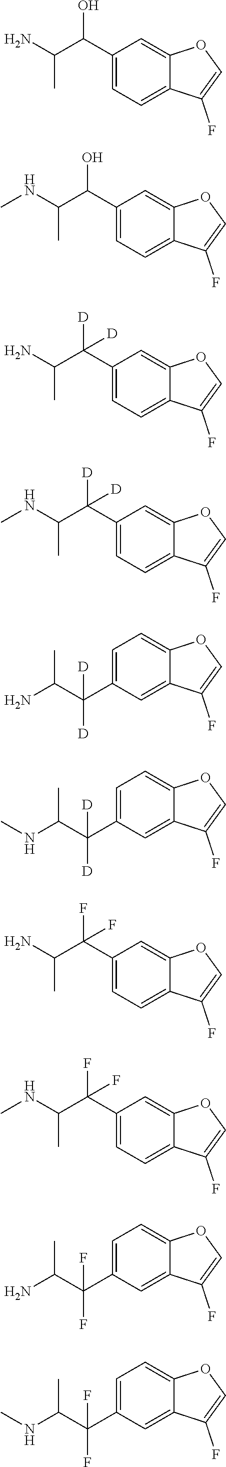

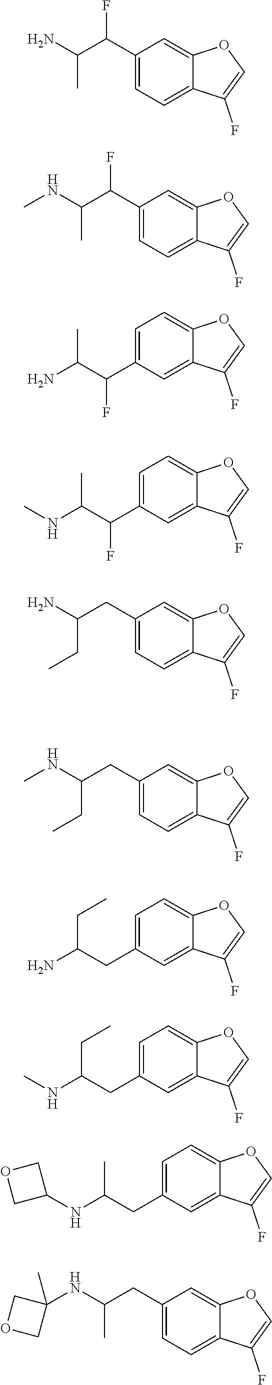

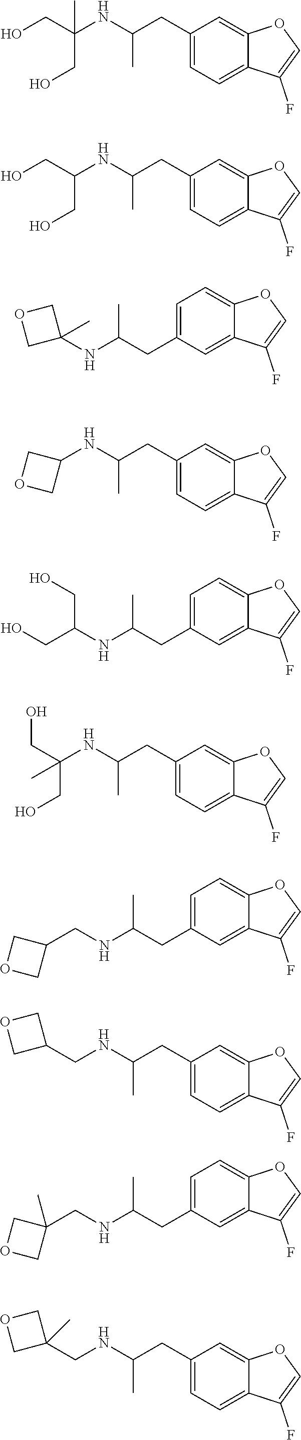

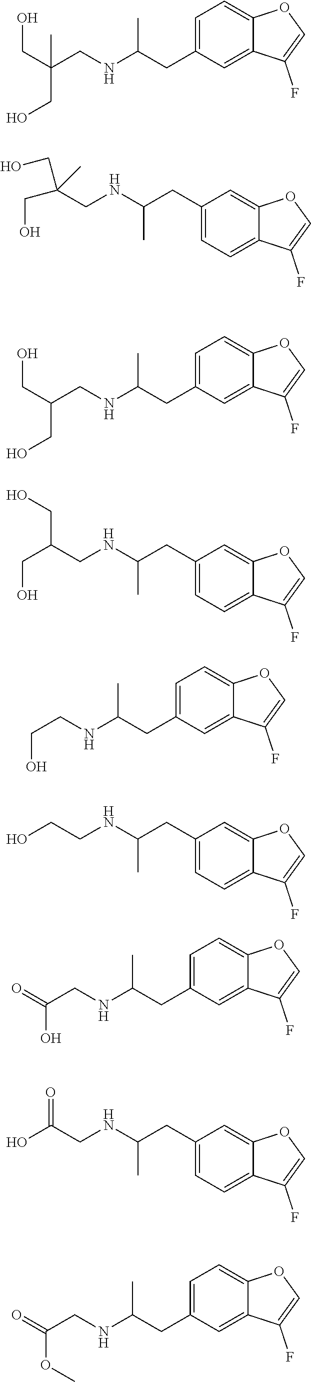

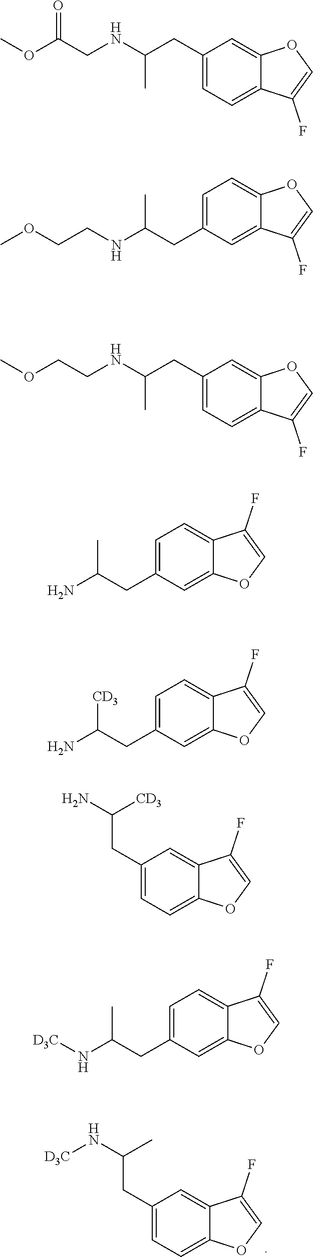

74. A composition comprising the compound of claim 1 and a pharmaceutically acceptable carrier.

75. A method of treating a neurological disorder, comprising administering to a subject in need thereof an effective amount of the compound of claim 1.

76. The method of claim 75, wherein the neurological disorder is post-traumatic stress disorder.

77. The method of claim 75, wherein the neurological disorder is an eating disorder including post-traumatic stress disorder.

78. The method of claim 77, wherein the neurological disorder is obsessive-compulsive disorder (OCD).
